(19) United States
(12) Patent Application Publication
(10) Pub. No.: US 2026/0232222 A1
(43) Pub. Date: Aug. 13, 2026

(54) DEVICE FOR MEASUREMENT COLLECTION AND TREATMENT

(71) Applicant: The Cleveland Clinic Foundation, Cleveland, OH (US)

(72) Inventors: Sean NAGEL, Cleveland Heights, OH (US); Stephen JONES, Cleveland Heights, OH (US); Hongchae BAEK, Cleveland, OH (US)

(21) Appl. No.: 19/159,466

(22) PCT Filed: Feb. 20, 2024
(86) PCT No.: PCT/US2024/016555

§ 371 Date: Aug. 25, 2025

Related U.S. Application Data
(60) Provisional application No. 63/447,006, filed on Feb. 20, 2023.

(51) Int. Cl. A61B 5/11; A61B 5/00; G16H 30/40

(52) U.S. Cl. CPC A61B 5/1101; A61B 5/002; A61B 5/0024; A61B 5/4082; A61B 5/4836; A61B 5/6802; G16H 30/40

(57) ABSTRACT

One or more wearable devices, computing devices, systems and/or methods are provided. In an example, a device is provided. The device may include one or more accelerometers configured to generate measurement data based upon movement associated with a body part of a person. The device may include a communication device configured to transmit a message comprising the measurement data to a computing device. The device may be operable within an imaging region of an imaging machine.

### RELATED APPLICATION

This application claims priority to U.S. Provisional Patent Application No. 63/447,006, filed on Feb. 20, 2023, entitled “Wireless Accelerometer Sleeve for MRI Guided Focused Ultrasound,” which is incorporated herein by reference in its entirety.

### TECHNICAL FIELD

The present disclosure relates to devices for collecting measurement data.

### BACKGROUND

Magnetic Resonance-guided Focused Ultrasound (MRgFUS) is a non-invasive treatment for essential tremor, tremor-dominant Parkinson's disease and other conditions. MRgFUS utilizes focused ultrasound waves to create thermal lesions in the thalamus, a region of the brain associated with motor control. The procedure is guided by real-time magnetic resonance imaging (MRI), allowing clinicians to visualize the targeted area and adjust the treatment parameters accordingly. By delivering focused ultrasound energy to specific neural pathways responsible for tremors, MRgFUS aims to interrupt abnormal signaling and alleviate symptoms without the need for surgical incisions or invasive procedures.

### SUMMARY

In accordance with the present disclosure, one or more wearable devices, computing devices, systems and/or methods are provided. In an example, a device is provided. The device may comprise (i) one or more accelerometers configured to generate measurement data based upon movement associated with a body part of a person, and/or (ii) a communication device configured to transmit a message comprising the measurement data to a computing device. The device may be operable within an imaging region of an imaging machine.

In an example, a method is provided. A wireless transmission of measurement data may be received from a wearable device. One or more operations may be performed on the measurement data to identify one or more tremors of a person.

In an example, a method is provided. A transmission of measurement data may be received from a device that is operable within an imaging region of an imaging machine. A treatment performed on a person using the imaging machine may be controlled based upon the measurement data.

In an example, a device is provided. The device may comprise (i) one or more accelerometers configured to generate measurement data based upon movement associated with a body part of a person, and/or (ii) an indicator device configured to output a treatment indication based upon the measurement data, wherein the treatment indication is usable to control a treatment of the person.

### DESCRIPTION OF THE DRAWINGS

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1 is an illustration of a scenario involving various examples of networks that may connect servers and clients.

FIG. 2 is an illustration of a scenario involving an example configuration of a server that may utilize and/or implement at least a portion of the techniques presented herein.

FIG. 3 is an illustration of a scenario involving an example configuration of a client that may utilize and/or implement at least a portion of the techniques presented herein.

FIG. 4A illustrates a device, in accordance with some embodiments.

FIG. 4B is a component block diagram illustrating a wearable device collecting measurement data and/or transmitting the measurement data to a client device, in accordance with some embodiments.

FIG. 4C is a component block diagram illustrating a wearable device collecting measurement data and/or transmitting the measurement data to a client device, in accordance with some embodiments.

FIG. 5A is a component block diagram illustrating a scenario in which a wearable device is used to collect measurement data in association with a treatment of a person, in accordance with some embodiments.

FIG. 5B illustrates a client device displaying a portion of a movement assessment interface, in accordance with some embodiments.

FIG. 5C illustrates a client device displaying a treatment control interface, in accordance with some embodiments.

FIG. 5D illustrates a client device displaying a portion of a movement assessment interface, in accordance with some embodiments.

FIG. 5E illustrates a client device displaying a portion of a movement assessment interface, in accordance with some embodiments.

FIG. 5F illustrates a client device displaying a portion of a movement assessment interface, in accordance with some embodiments.

FIG. 5G illustrates a client device displaying a portion of a movement assessment interface, in accordance with some embodiments.

FIG. 5H illustrates a client device displaying a portion of a movement assessment interface, in accordance with some embodiments.

FIG. 5I illustrates a client device displaying a portion of a movement assessment interface, in accordance with some embodiments.

FIG. 5J illustrates a client device displaying a portion of a movement assessment interface, in accordance with some embodiments.

FIG. 5K illustrates a client device displaying a portion of a movement assessment interface, in accordance with some embodiments.

FIG. 5L illustrates a client device displaying a portion of a movement assessment interface, in accordance with some embodiments.

FIG. 5M illustrates a client device displaying a portion of a movement assessment interface, in accordance with some embodiments.

FIG. 5N illustrates a client device displaying a portion of a movement assessment interface, in accordance with some embodiments.

FIG. 6 is a flow chart illustrating an example method, in accordance with some embodiments.

FIG. 7 is a flow chart illustrating an example method, in accordance with some embodiments.

FIG. 8A illustrates a device, in accordance with some embodiments.

FIG. 8B illustrates a device, in accordance with some embodiments.

FIG. 8C illustrates a device, in accordance with some embodiments.

FIG. 9A is a component block diagram illustrating a scenario in which a drafting test result is determined using an image associated with a drafting test, in accordance with some embodiments.

FIG. 9B illustrates data structures associated with drafting test results, in accordance with some embodiments.

FIG. 9C illustrates data structures associated with drafting test results, in accordance with some embodiments.

FIG. 9D illustrates data structures associated with drafting test results, in accordance with some embodiments.

FIG. 9E illustrates data structures associated with drafting test results, in accordance with some embodiments.

FIG. 9F is a component block diagram illustrating a scenario in which a tremor status is determined using a drafting test result and accelerometer-derived data, in accordance with some embodiments.

FIG. 9G illustrates data structures associated with tremor statuses, in accordance with some embodiments.

FIG. 10 is an illustration of a scenario featuring an example non-transitory machine readable medium in accordance with one or more of the provisions set forth herein.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems. Accordingly, this subject matter is not intended to be construed as limited to any example embodiments set forth herein. Rather, example embodiments are provided merely to be illustrative. Such embodiments may, for example, take the form of hardware, software, firmware, medicine, clothing design, or any combination thereof.

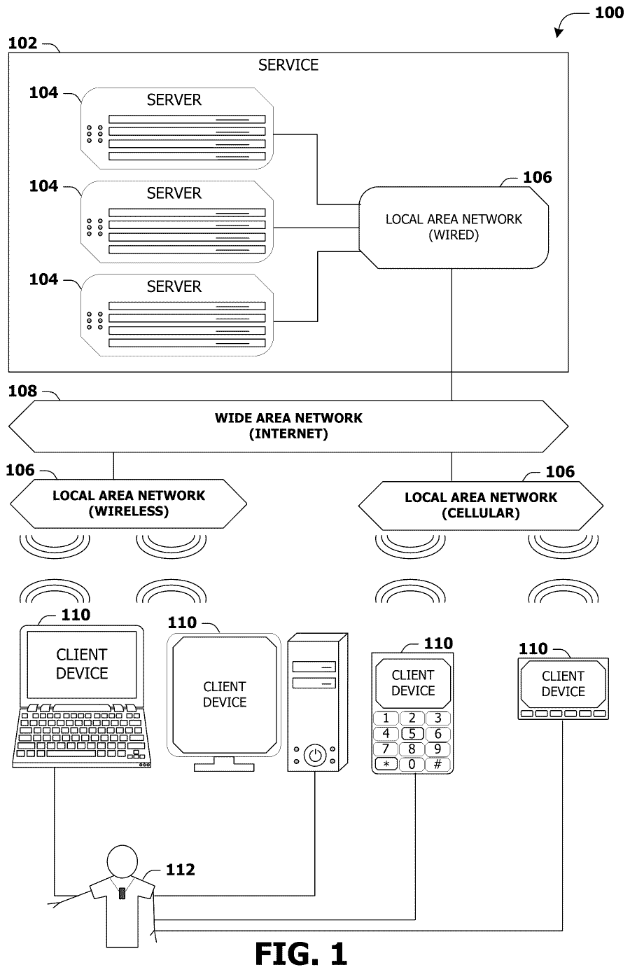

FIG. 1 is an interaction diagram of a scenario 100 illustrating a service 102 provided by a set of servers 104 to a set of client devices 110 via various types of networks. The servers 104 and/or client devices 110 may be capable of transmitting, receiving, processing, and/or storing many types of signals, such as in memory as physical memory states.

In the scenario 100 of FIG. 1, the service 102 may be accessed via a wide area network 108 (WAN) by a user 112 of one or more client devices 110, such as a portable media player (e.g., an electronic text reader, an audio device, or a portable gaming, exercise, or navigation device); a portable communication device (e.g., a camera, a phone, a wearable or a text chatting device); a workstation; and/or a laptop form factor computer. The respective client devices 110 may communicate with the service 102 via various connections to the wide area network 108.

One or more client devices 110 may comprise a cellular communicator and may communicate with the service 102 by connecting to the wide area network 108 via a wireless local area network 106 (LAN) provided by a cellular provider.

Alternatively and/or additionally, one or more client devices 110 may communicate with the service 102 by connecting to the wide area network 108 via a wireless local area network 106 provided by a location such as the user's home or workplace. The wireless local area network 106 may, for example, be a WiFi (Institute of Electrical and Electronics Engineers (IEEE) Standard 802.11) network or a Bluetooth (IEEE Standard 802.15.1) personal area network.

It may be appreciated that the servers 104 and the client devices 110 may communicate over various types of networks. Exemplary types of networks that may be accessed by the servers 104 and/or client devices 110 include mass storage, such as network attached storage (NAS), a storage area network (SAN), or other forms of computer or machine readable media.

The servers 104 of the service 102 may be interconnected directly, or through one or more other networking devices, such as routers, switches, and/or repeaters. The servers 104 may utilize a variety of physical networking protocols, such as Ethernet and/or Fiber Channel, and/or logical networking protocols, such as variants of an Internet Protocol (IP), a Transmission Control Protocol (TCP), and/or a User Datagram Protocol (UDP).

The servers 104 of the service 102 may be internally connected via a local area network 106. The local area network 106 may be organized according to one or more network architectures, such as server/client, peer-to-peer, and/or mesh architectures, and/or a variety of roles, such as administrative servers, authentication servers, security monitor servers, data stores for objects such as files and databases, business logic servers, time synchronization servers, and/or front-end servers providing a user-facing interface for the service 102.

The local area network 106 may be a wired network where network adapters on the respective servers 104 are interconnected via cables (e.g., coaxial and/or fiber optic cabling), and may be connected in various topologies (e.g., buses, token rings, meshes, and/or trees). The local area network 106 may include, e.g., analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art.

Alternatively and/or additionally, the local area network 106 may comprise one or more sub-networks, such as may employ differing architectures, may be compliant or compatible with differing protocols and/or may interoperate within the local area network 106. Additionally, a variety of local area networks 106 may be interconnected; e.g., a router may provide a link between otherwise separate and independent local area networks 106.

In the scenario 100 of FIG. 1, the local area network 106 of the service 102 is connected to a wide area network 108 that allows the service 102 to exchange data with other services 102 and/or client devices 110. The wide area network 108 may encompass various combinations of devices with varying levels of distribution and exposure, such as a public wide-area network (e.g., the Internet) and/or a private network (e.g., a virtual private network (VPN) of a distributed enterprise).

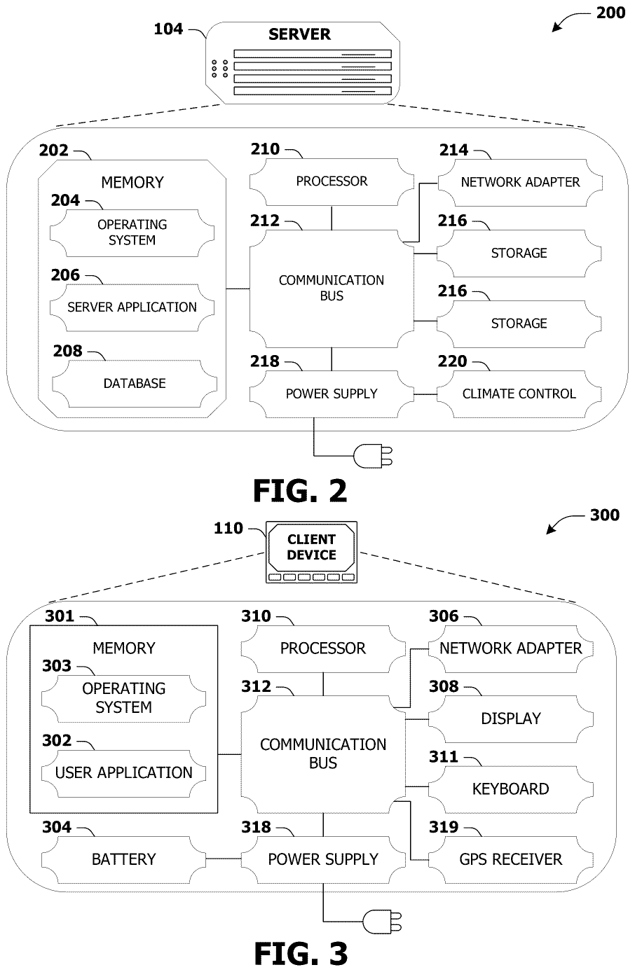

FIG. 2 presents a schematic architecture diagram 200 of a server 104 that may utilize at least a portion of the techniques provided herein. Such a server 104 may vary widely in configuration or capabilities, alone or in conjunction with other servers, in order to provide a service such as the service 102.

The server 104 may comprise a variety of peripheral components, such as a wired and/or wireless network adapter 214 connectible to a local area network and/or wide area network; one or more storage components 216, such as a hard disk drive, a solid-state storage device (SSD), a flash memory device, and/or a magnetic and/or optical disk reader.

The server 104 may comprise memory 202 storing various forms of applications, such as an operating system 204; one or more server applications 206, such as a hypertext transport protocol (HTTP) server, a file transfer protocol (FTP) server, or a simple mail transport protocol (SMTP) server; and/or various forms of data, such as a database 208 or a file system.

The server 104 may comprise one or more processors 210 that process instructions. The one or more processors 210 may optionally include a plurality of cores; one or more coprocessors, such as a mathematics coprocessor or an integrated graphical processing unit (GPU); and/or one or more layers of local cache memory.

The server 104 may comprise a mainboard featuring one or more communication buses 212 that interconnect the processor 210, the memory 202, and various peripherals, using a variety of bus technologies, such as a variant of a serial or parallel AT Attachment (ATA) bus protocol; a Uniform Serial Bus (USB) protocol; and/or Small Computer System Interface (SCI) bus protocol. In a multibus scenario, a communication bus 212 may interconnect the server 104 with at least one other server.

The server 104 may operate in various physical enclosures, such as a desktop or tower, and/or may be integrated with a display as an “all-in-one” device. The server 104 may be mounted horizontally and/or in a cabinet or rack, and/or may simply comprise an interconnected set of components.

The server 104 may provide power to and/or receive power from another server and/or other devices. The server 104 may comprise a dedicated and/or shared power supply 218 that supplies and/or regulates power for the other components. The server 104 may comprise a shared and/or dedicated climate control unit 220 that regulates climate properties, such as temperature, humidity, and/or airflow.

The server 104 may include one or more other components that are not shown in the schematic diagram 200 of FIG. 2, such as a display; a display adapter, such as a graphical processing unit (GPU); input peripherals, such as a keyboard and/or mouse; and a flash memory device that may store a basic input/output system (BIOS) routine that facilitates booting the server 104 to a state of readiness. A plurality of such servers 104 may be configured and/or adapted to utilize at least a portion of the techniques presented herein.

FIG. 3 presents a schematic architecture diagram 300 of a client device 110 whereupon at least a portion of the techniques presented herein may be implemented. Such a client device 110 may vary widely in configuration or capabilities, in order to provide a variety of functionality to a user such as the user 112.

The client device 110 may comprise memory 301 storing various forms of applications, such as an operating system 303; one or more user applications 302, such as document applications, media applications, file and/or data access applications, communication applications such as web browsers and/or email clients, utilities, and/or games; and/or drivers for various peripherals.

In some examples, as a user 112 interacts with a software application on a client device 110 (e.g., an instant messenger and/or electronic mail application), descriptive content in the form of signals or stored physical states within memory (e.g., an email address, instant messenger identifier, phone number, postal address, message content, date, and/or time) may be identified.

In such examples, descriptive content may be stored, typically along with contextual content. For example, the source of an email address (e.g., a communication received from another user via an instant messenger application) may be stored as contextual content associated with the email address. Contextual content, therefore, may identify circumstances surrounding receipt of an email address (e.g., the date or time that the email address was received), and may be associated with descriptive content. Contextual content, may, for example, be used to subsequently search for associated descriptive content. For example, a search for email addresses received from specific individuals, received via an instant messenger application or at a given date or time, may be initiated.

The client device 110 may comprise one or more processors 310 that process instructions. The one or more processors 310 may optionally include a plurality of cores; one or more coprocessors, such as a mathematics coprocessor or an integrated graphical processing unit (GPU); and/or one or more layers of local cache memory.

The client device 110 may comprise a dedicated and/or shared power supply 318 that supplies and/or regulates power for other components, and/or a battery 304 that stores power for use while the client device 110 is not connected to a power source via the power supply 318. The client device 110 may provide power to and/or receive power from other client devices.

The client device 110 may comprise a variety of peripheral components, such as a wired and/or wireless network adapter 306 connectible to a local area network and/or wide area network; one or more output components, such as a display 308 coupled with a display adapter (optionally including a graphical processing unit (GPU)), a sound adapter coupled with a speaker, and/or a printer; input devices for receiving input from the user, such as a keyboard 311, a mouse, a microphone, a camera, and/or a touch-sensitive component of the display 308; and/or environmental sensors, such as a global positioning system (GPS) receiver 319 that detects the location, velocity, and/or acceleration of the client device 110, a compass, accelerometer, and/or gyroscope that detects a physical orientation of the client device 110.

The client device 110 may comprise a mainboard featuring one or more communication buses 312 that interconnect the processor 310, the memory 301, and various peripherals, using a variety of bus technologies, such as a variant of a serial or parallel AT Attachment (ATA) bus protocol; the Uniform Serial Bus (USB) protocol; and/or the Small Computer System Interface (SCI) bus protocol.

The client device 110 may include one or more other components that are not shown in the schematic architecture diagram 300 of FIG. 3, such as one or more storage components, such as a hard disk drive, a solid-state storage device (SSD), a flash memory device, and/or a magnetic and/or optical disk reader; and/or a flash memory device that may store a basic input/output system (BIOS) routine that facilitates booting the client device 110 to a state of readiness. In some examples, the client device 110 may include a climate control unit that regulates climate properties, such as temperature, humidity, and airflow.

The client device 110 may include one or more servers that may locally serve the client device 110 and/or other client devices of the user 112 and/or other individuals. For example, a locally installed webserver may provide web content in response to locally submitted web requests. Many such client devices 110 may be configured and/or adapted to utilize at least a portion of the techniques presented herein.

The client device 110 may serve the user in a variety of roles, such as a workstation, kiosk, media player, gaming device, and/or appliance. The client device 110 may therefore be provided in a variety of form factors, such as a desktop or tower workstation; an “all-in-one” device integrated with a display 308; a laptop, tablet, convertible tablet, or palmtop device; a wearable device mountable in a headset, eyeglass, earpiece, and/or wristwatch, and/or integrated with an article of clothing; and/or a component of a piece of furniture, such as a tabletop, and/or of another device, such as a vehicle or residence.

One or more devices and/or techniques for collecting measurement data of a person and/or using the measurement data to control treatment parameters are provided. A wearable device may comprise one or more accelerometers positioned in one or more respective positons along one or more regions of the person's body. The one or more accelerometers may be supported in the one or more respective positions using at least one of a sleeve, a shirt, a turtleneck, and/or other mechanism. The wearable device may comprise a communication device (e.g., a wireless communication device and/or a wired communication device) that transmits measurement data collected by the one or more accelerometers over a connection (e.g., a wireless connection and/or a wired connection) to a client device. In some examples, measurement data collected using the wearable device and/or transmitted to the client device (and/or other client devices) may be used to determine a set of tremor features associated with the person, monitor the person and/or track tremor symptoms of the person over a period of time (e.g., short-term, long-term, etc.).

Alternatively and/or additionally, the measurement data and/or the set of tremor features may be used in conjunction with a treatment of the person, such as a Magnetic Resonance-Guided Focused Ultrasound (MRgFUS) treatment and/or a deep brain stimulation (DBS) treatment performed to alleviate tremor symptoms of the person. For example, a treatment control system may use the measurement data and/or the set of tremor features to provide guidance, predictions and/or suggestions to modify a parameter (e.g., energy level, target region, etc.) of the treatment (e.g., the MRgFUS treatment and/or the DBS treatment). Alternatively and/or additionally, the client device may provide content indicative of the measurement data and/or the set of tremor features to a remote client device for display to a remote healthcare professional (e.g., a physician, surgeon, nurse, etc. tasked with managing and/or controlling one or more aspects of the treatment of the person).

It may be appreciated that by enabling the wearable device to communicate wirelessly (e.g., equipping the wearable device with the wireless communication device), the wearable device can be used more conveniently in a variety of environments (e.g., at home, at a place of work, during treatment, etc.) for tracking tremors of the person short-term (e.g., over the period of one or more hours and/or days) and/or long-term (e.g., over the period of one or more weeks and/or months), as compared with an implementation where the wearable device relies on a wired connection to transfer collected measurement data to a device.

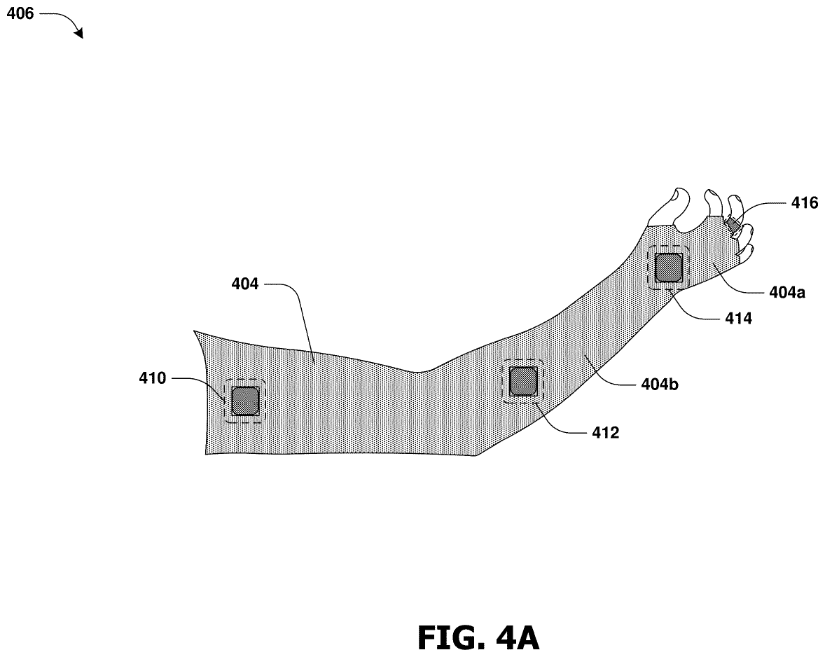

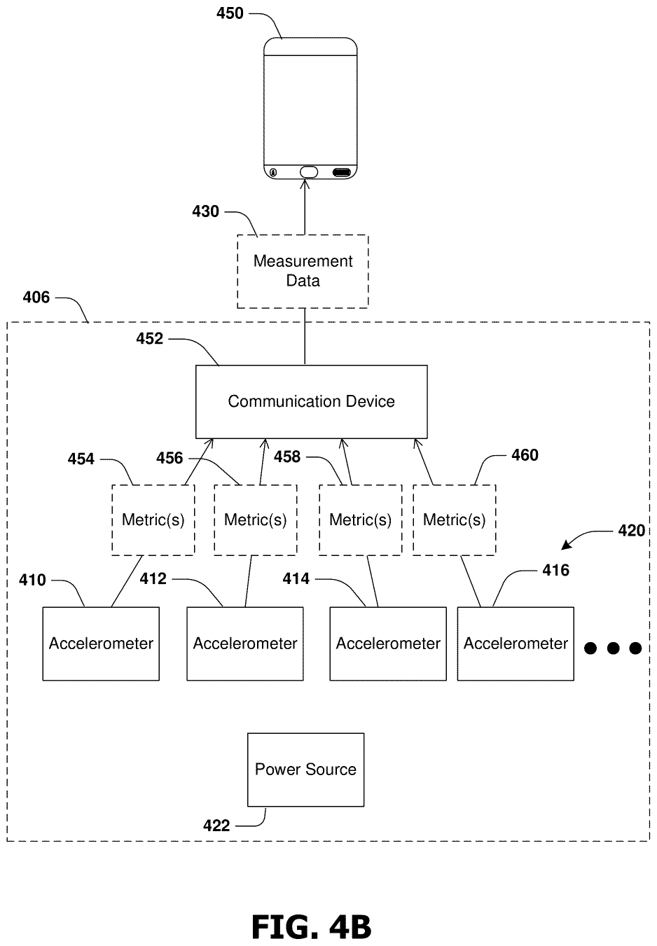

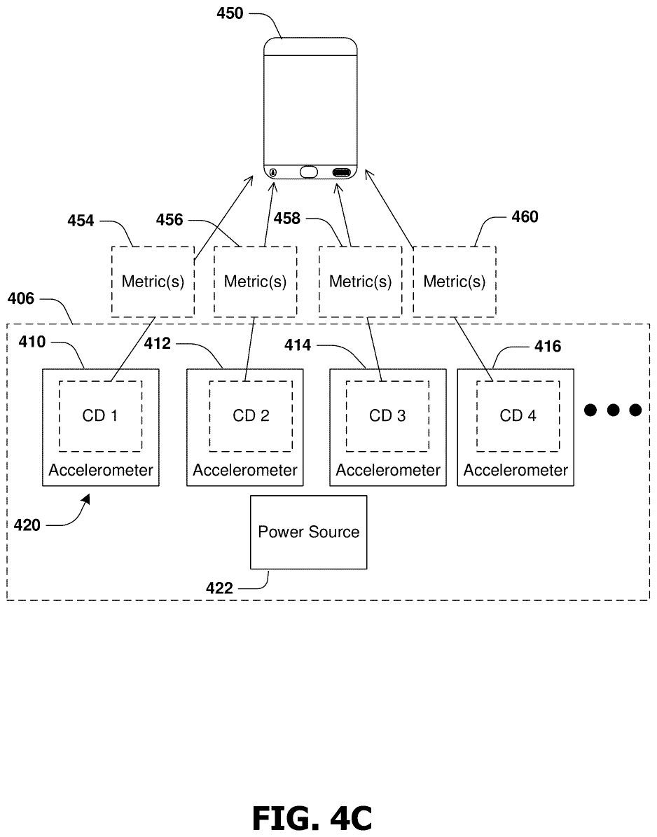

FIG. 4A illustrates a device 406 according to some embodiments. In some examples, the device 406 may comprise a sleeve 404 and/or a set of accelerometers (e.g., a set of one or more accelerometers). The set of accelerometers may comprise a first accelerometer 410, a second accelerometer 412, a third accelerometer 414 and/or a fourth accelerometer 416. The sleeve 404 may surround at least a portion of an arm of a person. For example, the sleeve 404 may surround (and/or may be in contact with) about 10% of a surface of the arm, between about 10% and about 90% of the surface of the arm, and/or between about 90% and about 100% of the surface of the arm (and/or an entirety of the arm). In some examples, the sleeve 404 may comprise one or more openings for one or more of the person's fingers to extend through. The set of accelerometers may be attached to the sleeve 404 and/or at least partially embedded in the sleeve 404 (e.g., embedded in a material of the sleeve 404). In an example, an accelerometer may be in a pocket (e.g., a sealed pocket or an unsealed pocket) of the sleeve 404. Alternatively and/or additionally, the accelerometer may be between a first layer of the sleeve 404 and a second layer of the sleeve 404.

In some examples, the sleeve 404 may be a form-fitting sleeve, which may follow (e.g., tightly follow) contours of the arm of the person and/or may apply pressure to support an accelerometer of the set of accelerometers in a target position (e.g., the sleeve 404 may press the accelerometer against a desired region of the arm) such that the accelerometer can capture target metrics. The accelerometer may be contact with the desired region of the arm. For example, the accelerometer may be in direct contact with the desired region of the arm. Alternatively and/or additionally, the accelerometer may be in indirect contact with the desired region of the arm (e.g., a layer, such as a layer of the sleeve 404, may be between the accelerometer and the desired region of the arm). In an example, the sleeve 404 may comprise cotton, synthetic fiber, one or more polymers, and/or other material. In some examples, the sleeve 404 may be a standalone sleeve (e.g., the sleeve 404 may not be part of a larger piece of clothing, such as a shirt and/or gown). Alternatively and/or additionally, the sleeve 404 may be a part of a shirt (e.g., a long sleeve shirt, a turtleneck, etc.), a gown, or other article of clothing. Other features and/or mechanisms (other than the sleeve 404) of the device 406 for supporting one or more accelerators of the set of accelerometers in one or more respective positions (along the arm and/or other region of the person, for example) are within the scope of the present disclosure.

In some examples, a first portion 404a of the sleeve 404 comprises one or more first materials and a second portion 404b of the sleeve 404 comprises one or more second materials. The first portion 404a of the sleeve 404 may at least partially surround the person's hand. In some examples, the one or more first materials are softer and/or more flexible than the one or more second materials, which may allow the person's hand to be moved and/or perform tasks (e.g., move fingers, hold an object such as a cup or pen, etc.) with increased ease while the sleeve 404 is worn. In some examples, the one or more first materials comprise at least one of rubber, mesh, etc. The one or more first materials may be embedded within the first portion 404a of the sleeve 404. In some examples, the first portion 404a may comprise one or more one or more gripping features to enhance grip performance of the sleeve 404.

In some examples, multiple disposable and/or reusable sleeves (and/or other types of clothing, such as form-fitting clothing) may be provided to the person (for long-term monitoring, for example) such that the person can switch out a used sleeve for a new sleeve (and/or a cleaned used sleeve) when the used sleeve has been used for over a threshold amount of time, is in a deteriorated condition, and/or needs to be cleaned and/or refurbished before subsequent use.

In some examples, an accelerometer of the set of accelerometers (e.g., the first accelerometer 410, the second accelerometer 412, the third accelerometer 414 and/or the fourth accelerometer 416) may comprise a 9-axis accelerometer. In an example, the accelerometer (e.g., the 9-axis accelerometer) may be a device (e.g., a micro-electromechanical system (MEMS) motion sensor) comprising (i) a linear acceleration device (e.g., 3-axis linear acceleration device for measuring linear acceleration), (ii) a rotational acceleration/velocity device (e.g., 3-axis rotational acceleration/velocity device for measuring rotational acceleration and/or rotational velocity), and/or (iii) a magnetometer (e.g., a 3-axis magnetometer). Alternatively and/or additionally, the accelerometer may comprise a gyroscope and/or a compass. Using a 9-axis accelerometer may provide for improved and/or more accurate measurements determined by the accelerometer, as compared with other types of accelerometers (e.g., 3-axis accelerometer, 6-axis accelerometer, etc.). Other types of the accelerometer (e.g., 3-axis accelerometer, 6-axis accelerometer, or other type of accelerometer) of the set of accelerometers are within the scope of the present disclosure. In some examples, an accelerometer of the set of accelerometers (e.g., the first accelerometer 410, the second accelerometer 412, the third accelerometer 414 and/or the fourth accelerometer 416) may be configured to determine multi-modal measurements (e.g., at least one of frequency, amplitude, angle, irregularity, variability, jitter, etc.).

FIG. 4B illustrates communication of the device 406 with a first client device 450. The first client device 450 may comprise a phone, a tablet, a laptop, a computer, a wearable device, a smart device, a television, any other type of computing device, hardware and/or software. In some examples, the device 406 comprises a communication device 452. The communication device 452 may comprise a wireless communication device and/or a wired communication device. The wireless communication device may collect data (e.g., measurement data) from the set of accelerometers (shown with reference number 420) and/or may transmit (e.g., may broadcast and/or telecast) the data to the first client device 450. For example, the wireless communication device 452 may transmit a message 430, comprising at least some of the data, to the first client device 450. In some examples in which the communication device 452 comprises the wireless communication device, the device 406 is not connected via wire to the first client device 450 and/or transmits the message 430 over a wireless connection. In some examples in which the communication device 452 comprises the wired communication device, the device 406 is connected via a wired connection to the first client device 450 and/or transmits the message 430 over the wired connection.

In some examples, the first accelerometer 410 may provide one or more first metrics 454 to the communication device 452 (via a wired connection between the first accelerometer 410 and the communication device 452, for example). The first accelerometer 410 may be proximal a first region of the arm. In some examples, the first region of the arm may correspond to a region between a shoulder of the person and an elbow of the person. For example, the first accelerometer 410 may be in contact with (e.g., in direct contact with or in indirect contact with) the first region of the arm. The one or more first metrics 454 may be indicative of (e.g., may comprise and/or may be usable to determine) (i) an acceleration of motion of the first region of the arm and/or the first accelerometer 410, (ii) a vibration level of the first region of the arm and/or the first accelerometer 410, (iii) a displacement level (e.g., an amplitude of displacement) of the first region of the arm and/or the first accelerometer 410, (iv) a position of the first region of the arm and/or the first accelerometer 410, (v) a velocity of motion of the first region of the arm and/or the first accelerometer 410, (vi) a direction of motion of the first region of the arm and/or the first accelerometer 410, (vii) a frequency of movement of the first region of the arm and/or the first accelerometer 410, (viii) an angular velocity of the first region of the arm and/or the first accelerometer 410, and/or (ix) one or more other metrics.

In some examples, the second accelerometer 412 may provide one or more second metrics 456 to the communication device 452 (via a wired connection between the second accelerometer 412 and the communication device 452, for example). The second accelerometer 412 may be proximal a second region of the arm. In some examples, the second region of the arm may correspond to a forearm of the arm, such as a region between an elbow of the person and a wrist of the person. For example, the second accelerometer 412 may be in contact with (e.g., in direct contact with or in indirect contact with) the second region of the arm. The one or more second metrics 456 may be indicative of (e.g., may comprise and/or may be usable to determine) (i) an acceleration of motion of the second region of the arm and/or the second accelerometer 412, (ii) a vibration level of the second region of the arm and/or the second accelerometer 412, (iii) a displacement level (e.g., an amplitude of displacement) of the second region of the arm and/or the second accelerometer 412, (iv) a position of the second region of the arm and/or the second accelerometer 412, (v) a velocity of motion of the second region of the arm and/or the second accelerometer 412, (vi) a direction of motion of the second region of the arm and/or the second accelerometer 412, (vii) a frequency of movement of the second region of the arm and/or the second accelerometer 412, (viii) an angular velocity of the second region of the arm and/or the second accelerometer 412, and/or (ix) one or more other metrics.

In some examples, the third accelerometer 414 may provide one or more third metrics 458 to the communication device 452 (via a wired connection between the third accelerometer 414 and the communication device 452, for example). The third accelerometer 414 may be proximal a third region of the arm. In some examples, the third region of the arm may correspond to a wrist of the arm. For example, the third accelerometer 414 may be in contact with (e.g., in direct contact with or in indirect contact with) the third region of the arm. The one or more third metrics 458 may be indicative of (e.g., may comprise and/or may be usable to determine) (i) an acceleration of motion of the third region of the arm and/or the third accelerometer 414, (ii) a vibration level of the third region of the arm and/or the third accelerometer 414, (iii) a displacement level (e.g., an amplitude of displacement) of the third region of the arm and/or the third accelerometer 414, (iv) a position of the third region of the arm and/or the third accelerometer 414, (v) a velocity of motion of the third region of the arm and/or the third accelerometer 414, (vi) a direction of motion of the third region of the arm and/or the third accelerometer 414, (vii) a frequency of movement of the third region of the arm and/or the third accelerometer 414, (viii) an angular velocity of the third region of the arm and/or the third accelerometer 414, and/or (ix) one or more other metrics.

In some examples, the fourth accelerometer 416 may provide one or more fourth metrics 460 to the communication device 452 (via a wired connection between the fourth accelerometer 416 and the communication device 452, for example). The fourth accelerometer 416 may be proximal a fourth region of the arm. In some examples, the fourth region of the arm may correspond to a finger (e.g., middle finger and/or other finger) and/or other location of the person's hand. For example, the fourth accelerometer 416 may be in contact with (e.g., in direct contact with or in indirect contact with) the fourth region of the arm. The one or more fourth metrics 460 may be indicative of (e.g., may comprise and/or may be usable to determine) (i) an acceleration of motion of the fourth region of the arm and/or the fourth accelerometer 416, (ii) a vibration level of the fourth region of the arm and/or the fourth accelerometer 416, (iii) a displacement level (e.g., an amplitude of displacement) of the fourth region of the arm and/or the fourth accelerometer 416, (iv) a position of the fourth region of the arm and/or the fourth accelerometer 416, (v) a velocity of motion of the fourth region of the arm and/or the fourth accelerometer 416, (vi) a direction of motion of the fourth region of the arm and/or the fourth accelerometer 416, (vii) a frequency of movement of the fourth region of the arm and/or the fourth accelerometer 416, (viii) an angular velocity of the fourth region of the arm and/or the fourth accelerometer 416, and/or (ix) one or more other metrics.

In some examples, the set of accelerometers 420 may comprise one or more other accelerometers in addition, or as an alternative, to one or more of the accelerometers 410, 412, 414 and/or 416 shown in FIG. 4A. The one or more other accelerometers may be positioned proximal one or more respective regions of the person, such as at least one of the person's chest, shoulder, head, neck, midsection, stomach, leg, foot, one or more joints, one or more regions of the person's body that are adjacent to and/or surround one or more joints, etc. In some examples, accelerometers of the set of accelerometers 420 may be positioned proximal regions of a set of regions (of the person's body), respectively. The set of regions may comprise the first region, the second region, the third region, the fourth region, and/or one or more regions corresponding to at least one of the person's chest, shoulder, head, neck, midsection, stomach, leg, foot, one or more joints, one or more regions of the person's body that are adjacent to and/or surround one or more joints, etc.

In some examples, the message 430 (transmitted by the communication device 452 to the first client device 450) may be indicative of (e.g., may comprise and/or may be usable to determine) the one or more first metrics 454 from the first accelerometer 410, the one or more second metrics 456 from the second accelerometer 412, the one or more third metrics 458 from the third accelerometer 414, the one or more fourth metrics 460 from the fourth accelerometer 416, and/or one or more other metrics from one or more other accelerometers.

In some examples, the set of accelerometers 420 and/or the communication device 452 may be powered using a power source 422 (e.g., a battery). Embodiments are contemplated in which each accelerometer of one, some and/or all of the set of accelerometers 420 comprises (and/or is connected to) a respective power source (of a plurality of respective power sources) to power the accelerometer. In some examples, the power source 422 may be charged using a charging port. In some examples, the power source 422 and/or the charging port may be attached to the sleeve 404 and/or at least partially embedded in the sleeve 404 (e.g., embedded in a material of the sleeve 404). In some examples, the power source 422 and/or the charging port may be positioned proximal the person's hand.

In some examples, the device 406 comprises one or more switches (not shown) connected to the power source 422, the set of accelerometers 420, and/or the communication device 452. The one or more switches may comprise a switch (e.g., a single switch) to power all of set of accelerometers 420 and/or the communication device 452 (e.g., all of the set of accelerometers 420 and/or the communication device 452 may be turned on or off using the single switch). Alternatively and/or additionally, the one or more switches may comprise a plurality of switches comprising respective switches for powering respective accelerometers of the set of accelerometers 420. In some examples, the one or more switches may be attached to the sleeve 404 and/or at least partially embedded in the sleeve 404 (e.g., embedded in a material of the sleeve 404).

In some examples, the device 406 comprises a sensor (e.g., a proximity sensor, a motion sensor, etc.) configured to control the one or more switches (for powering the set of accelerometers 420 and/or the communication device 452, for example). In some examples, the sensor may detect when the device 406 is worn by the person and/or when the device 406 is not worn by the person. The sensor may power the set of accelerometers 420 and/or the communication device 452 in response to determining that the device 406 is worn by the person (and/or in response to the sleeve 404 being moved into a position surrounding the person's arm). The sensor may turn off the set of accelerometers 420 and/or the communication device 452 in response to determining that the device 406 is not worn by the person (and/or in response to the sleeve 404 being moved out of the position surrounding the person's arm). In some examples, the sensor may be attached to the sleeve 404 and/or at least partially embedded in the sleeve 404 (e.g., embedded in a material of the sleeve 404).

The communication device 452 (e.g., the wireless communication device) may transmit the message 430 to the first client device 450 via Bluetooth, Bluetooth Low Energy (BLE) and/or other wireless communication technology standard. The communication device 452 may establish a wireless connection (e.g., a Bluetooth connection and/or other type of connection) with the first client device 450, and/or may transmit the message 430 over the wireless connection. In some examples, the communication device 452 may transmit the message 430 over a paired transmission (while the communication device 452 and the first client device 450 are paired with each other, for example).

Embodiments are contemplated in which each accelerometer of one, some and/or all of the set of accelerometers 420 comprises (and/or is connected to) a respective communication device (of a plurality of respective communication devices) that is used to transmit measurement data derived using the accelerometer to the first client device 450. FIG. 4C illustrates a scenario in which each of at least some accelerometers of the device 406 comprises a respective communication device to transmit measurement data to the first client device 450. In an example, the first accelerometer 410 may comprise a first communication device CD 1 that transmits (over a wireless connection or a wired connection, for example) a message indicative of the one or more first metrics 454 to the first client device 450. The second accelerometer 412 may comprise a second communication device CD 2 that transmits (over a wireless connection or a wired connection, for example) a message indicative of the one or more second metrics 456 to the first client device 450. The third accelerometer 414 may comprise a third communication device CD 3 that transmits (over a wireless connection or a wired connection, for example) a message indicative of the one or more third metrics 458 to the first client device 450. The fourth accelerometer 416 may comprise a fourth communication device CD 4 that transmits (over a wireless connection or a wired connection, for example) a message indicative of the one or more fourth metrics 460 to the first client device 450.

In some examples, measurement data from the set of accelerometers 420 may be used for (i) diagnosing the person (e.g., determining a disease and/or a stage of the disease associated with the person), (ii) determining a prognosis of the person, (iii) treating the disease (e.g., determining a treatment plan for the person and/or controlling treatment parameters of a treatment for the person), and/or (iv) one or more other actions. Examples of the disease may include a neurological disease, a psychiatric disorder, a brain disease, a tumor (e.g., a brain tumor), essential tremor, Parkinson's disease (e.g., tremor-dominant Parkinson's disease), obsessive-compulsive disorder (OCD), epilepsy, neuropathic pain, etc.

A treatment system and/or the first client device 450 may perform one or more operations on the measurement data to identify one or more tremors associated with the person (e.g., the person may have a disease associated with tremors, such as essential tremor, Parkinson's disease, etc.). In some examples, the one or more operations may be performed using an application (e.g., a mobile application, a web application, etc.) provided by the treatment system. The treatment system may provide the first client device 450 with a resource (e.g., at least one of a hyperlink, a configuration file, executable code, etc.) for at least one of accessing, installing, configuring, running, etc. the application on the first client device 450. In some examples, operations of the application may be performed on the first client device 450 and/or on one or more servers of the treatment system (e.g., the application of the treatment system may be implemented at least partially on the one or more servers and/or at least partially on the first client device 450).

The treatment system (and/or the first client device 450) may determine, based upon the measurement data, a set of movement features (e.g., a set of one or more movement features) associated with movement of one or more body parts of the person and/or the sleeve 404. The set of movement features may be usable to identify the disease (for a diagnosis, for example), and/or may be used for determining the prognosis of the person, treating the disease, and/or one or more other actions. The set of movement features may be indicative of a set of angles of one or more joints of the person, which may comprise elbow, knee, shoulder, wrist, one or more joints of hips of the person, one or more joints of the hand of the person, one or more joints of a finger of the person, and/or one or more other joints of the person. In some examples, the set of movement features may comprise a set of tremor features (e.g., a set of one or more tremor features) associated with the one or more tremors of the person. In some examples, the one or more tremors may be identified (and/or one or more characteristics of the one or more tremors may be determined) based upon the set of tremor features.

In an example, the set of movement features (and/or the set of tremor features) may comprise (i) the set of angles of the one or more joints of the person, (ii) a displacement level associated with the one or more tremors (e.g., an amplitude of a changing position of a body part associated with the one or more tremors), (iii) an acceleration of the one or more tremors, (iv) a frequency of the one or more tremors (e.g., an average quantity of tremors per unit of time), (v) an angular velocity of the tremor (e.g., an angular velocity with which a body part, such as the hand and/or the arm of the person, moves), (vi) an angle of the one or more tremors, (vii) a direction of motion associated with the tremor (e.g., a direction in which a body part, such as the hand and/or the arm of the person, moves) and/or (viii) one or more other features. In some examples, the angle of the tremor (and/or the set of angles of the one or more joints) may be determined based upon the angular velocity and/or the direction of motion.

In some examples, the set of movement features (and/or the set of tremor features) may comprise tremor features associated with one or more defined regions of the person. The one or more defined regions may comprise (i) one, some and/or all of the set of regions (proximal the set of accelerometers 420, for example) of the person's body, (ii) one or more joints of the person, (e.g., elbow, knee, shoulder, wrist, one or more joints of hips of the person, one or more joints of the hand of the person, one or more joints of a finger of the person, and/or one or more other joints of the person, (iii) one or more or more regions corresponding to at least one of the person's chest, shoulder, head, neck, midsection, stomach, leg, foot, one or more joints, one or more regions of the person's body that are adjacent to and/or surround one or more joints, etc. (iv) and/or one or more other regions. In some examples, for each region of one, some and/or all of the set of regions, the treatment system (and/or the first client device 450) may determine one or more movement features (e.g., tremor features to be included in the set of tremor features) comprising (i) an angle of a joint associated with the region, (ii) a displacement level associated with the region (e.g., an amplitude of a changing position of the region during the one or more tremors), (iii) an acceleration of the region, (iv) a frequency of the region, (v) a direction of motion of the region (vi) an angular velocity of the region, and/or (vii) one or more other features. In some examples, the set of movement features (and/or the set of tremor features) may comprise multi-modal measurements (e.g., at least one of frequency, amplitude, angle, irregularity, variability, jitter, etc.).

In some examples, the treatment system (and/or the first client device 450) may generate and/or display a movement assessment interface indicative of movement information associated with the person. In an example, the movement assessment interface may provide a tremor assessment report for the person. The movement assessment interface may comprise one or more graphical objects and/or text indicative of (i) whether the person exhibited one or more tremors (e.g., whether the device 406 detected any tremors while worn by the person), (ii) a tremor severity level associated with one or more detected tremors of the person, (iii) a tremor type of the one or more detected tremors, (iv) one, some and/or all of the set of movement features (and/or one, some and/or all of the set of tremor features), and/or (v) other information associated with movement of the person.

In some examples, the treatment system may determine the tremor severity level based upon the set of movement features (and/or the set of tremor features). For example, the treatment system may determine the tremor severity level to be “low” based upon a determination that a first tremor feature (e.g., at least one of a finger displacement level of one or more detected tremors, an acceleration of the one or more detected tremors, an angular velocity of the one or more detected tremors, etc.) of the set of tremor features is within a first range. Alternatively and/or additionally, the treatment system may determine the tremor severity level to be “high” based upon a determination that the first tremor feature of the set of tremor features is within a second range (e.g., a minimum value of the second range may be greater than a maximum value of the first range).

In some examples, the treatment system may determine the tremor type by comparing the set of movement features (and/or the set of tremor features) with one or more movement profiles associated with one or more tremor types (e.g., at least one of Essential tremor, Parkinsonian tremor, Dystonic tremor, Cerebellar tremor, Functional tremor, Physiologic tremor, Orthostatic tremor, etc.). For example, the treatment system may determine that the tremor type associated with the person is a first tremor type (e.g., Essential tremor) based upon a determination that the set of movement features (and/or the set of tremor features) at least partially match a first profile associated with the first tremor type (e.g., one or more features of the set of movement features may be within one or more respective ranges of the first profile, and/or features of the set of movement features may have relationships with one another that match one or more first feature interrelationships indicated by the first profile). Alternatively and/or additionally, the treatment system may determine that the tremor type associated with the person is a second tremor type (e.g., Parkinsonian tremor) based upon a determination that the set of movement features (and/or the set of tremor features) at least partially match a second profile associated with the second tremor type (e.g., one or more features of the set of movement features may be within one or more respective ranges of the second profile, and/or features of the set of movement features may have relationships with one another that match one or more second feature interrelationships indicated by the second profile).

In some examples, the treatment system may perform one or more acts discussed herein in association with a treatment (e.g., a radiological treatment and/or other type of treatment) of the person, wherein the one or more acts may comprise (i) monitoring metrics from the set of accelerometers 420, (ii) determining the set of movement features, and/or (iii) generating and/or displaying the movement assessment interface. For example, the treatment system may update (e.g., continuously and/or in a periodic and/or aperiodic manner) the set of movement features and/or the movement assessment interface (in real time, for example) in (i) one or more periods of time prior to the treatment (for determining a pre-treatment status of the person, such as a pre-treatment tremor severity level, for example), (ii) one or more periods of time during the treatment (for monitoring a status of the person during the treatment and/or for determining how the person is responding to the treatment to accurately control one or more parameters of the treatment, for example) (e.g., the device 406 may be used for aiding the treatment at multiple time points along the course of the treatment), and/or (iii) one or more periods of time after the treatment (to check for improvement or worsening of tremor symptoms and/or to monitor for a change, such as reduction, in tremor severity level of the person after the treatment relative to the pre-treatment tremor severity level, for example).

In some examples, the treatment system may present the movement assessment interface (e.g., tremor assessment interface) on a display (e.g., a display of the first client device 450 and/or other device). The movement assessment interface may be displayed to one or more healthcare professionals (e.g., physician, surgeon, nurse, etc.) that are associated with the treatment of the person. For example, the movement assessment interface (which may be updated to provide real-time representations of movement of the person, for example) may provide a healthcare professional with information about the person's response to the treatment (e.g., whether the person is improving, whether tremors are reducing or increasing, etc.) that can enable the healthcare professional to have an improved understanding of an effectiveness of the treatment thus far and/or make a more informed decision of one or more next steps of the treatment (as compared with some techniques that rely upon a healthcare professional to subjectively evaluate and/or assess tremor response in an on-site manner without using the device 406, which may lead to incomplete tremor control, non-durable improvement and/or undesirable side effects). Alternatively and/or additionally, the movement assessment interface may enable the one or more healthcare professionals to observe an impact (e.g., a real-time impact) of the treatment on the person, which may allow the one or more healthcare professionals to tailor one or more aspects of the treatment to the person based upon the observed impact.

In some examples, a treatment control interface may be displayed on the first client device 450 and/or a second client device (not shown). In some examples, the treatment control interface may comprise one or more selectable inputs for controlling (e.g., selecting and/or adjusting) one or more treatment parameters of a set of treatment parameters of the treatment. The treatment control interface may be used by a healthcare professional to control the set of treatment parameters of the treatment. In some examples, a set of updated treatment parameters (e.g., a set of one or more updated treatment parameters) of the treatment may be received via the treatment control interface. For example, the healthcare professional may submit an entry of the set of updated treatment parameters (e.g., the healthcare professional may select values of the set of updated treatment parameters) using the treatment control interface. For example, the treatment system may comprise a treatment control system to control (e.g., advise, influence, modify, tailor, aid, facilitate, etc.) the treatment based upon the set of updated treatment parameters. For example, the set of treatment parameters may comprise one or more parameters of one or more machines used to perform the treatment. The treatment control system may control the one or more machines based upon the set of updated treatment parameters.

In some examples, the one or more healthcare professionals include a remote healthcare professional located apart from a location at which the person is physically treated. The remote healthcare professional may be at least one of a physician, surgeon, nurse, etc. tasked with administering and/or managing the treatment. The remote healthcare professional may be located at a remote medical treatment site in a different location (e.g., a different geographical region, a different building, a different city, a different state, etc.) than the location at which the person is physically treated. In an example, the treatment may be performed on the person at a first healthcare facility, while the remote healthcare professional can control the treatment remotely from the remote medical treatment site. The remote medical treatment site may correspond to (i) a second healthcare facility different than the first healthcare facility, (ii) a room in the first healthcare facility that is separate from a room in which the treatment is performed on the person, (iii) a home and/or place of work of the remote healthcare professional, and/or (iv) other location. In some examples, the movement assessment interface may be displayed on a remote client device (e.g., a phone, a tablet, a laptop, a computer, a wearable device, a smart device, a television, any other type of computing device, hardware and/or software) associated with the remote medical treatment site and/or the remote healthcare professional (e.g., the movement assessment interface may be visible to the remote healthcare professional when the movement assessment interface is displayed on the remote client device).

In some examples, the treatment system (and/or the first client device 450) may transmit content (e.g., content of the movement assessment interface), that is indicative of at least some of the measurement data and/or the set of movement features, to a remote network associated with the remote medical treatment site. For example, the remote network may comprise a network of one or more healthcare professional devices comprising the remote client device. The content may be delivered to the remote client device via the remote network. The content (e.g., real-time content indicative of measurement data and/or movement features that are updated in at least one of a continuous manner, a periodic manner, and/or an aperiodic manner) may be rendered on a display of the remote client device to produce the movement assessment interface. In some examples, displaying the movement assessment interface on the remote client device may enable the remote healthcare professional to observe an impact (e.g., a real-time impact) of the treatment on the person, which may allow the remote healthcare professional to tailor one or more aspects of the treatment to the person based upon the observed impact. In some examples, the treatment control interface may be displayed on the remote client device (and/or a second remote client device associated with the remote healthcare professional) to enable the remote healthcare professional to remotely control one or more parameters of the treatment. For example, the set of updated treatment parameters (e.g., a set of one or more updated treatment parameters) of the treatment may be received from the remote network (e.g., the set of updated treatment parameters may be based upon an entry submitted by the remote healthcare professional via the treatment control interface). Thus, one or more of the techniques provided herein may be used to perform remote treatment (e.g., remote surgeries).

In some examples, the first client device 450 may be configured to send (e.g., via email or other communication platforms) to one or more other computing devices, such as to a healthcare provider, content (e.g., content of the movement assessment interface), that is indicative of at least some of the measurement data and/or the set of movement features.

Alternatively and/or additionally, the movement assessment interface may comprise a treatment guide indicative of one or more subsequent treatment acts of the treatment. The treatment system may determine the one or more subsequent treatment acts based upon the set of movement features. For example, the treatment system may use metrics from the set of accelerometers 420 (and/or the set of movement features determined based upon the metrics) as feedback of the treatment for use in determining the one or more subsequent treatment acts of the treatment. The treatment system may update the treatment guide in response to new information (e.g., real-time metrics from the set of accelerometers 420 and/or movement features determined based upon the real-time metrics).

In some examples, the message 430 (and/or one or more other messages carrying one or more metrics of one or more accelerometers of the set of accelerometers 420) is usable to control one or more machines (e.g., an imaging machine, an ultrasound device and/or one or more other machines) that are used to perform the treatment on the person. For example, the treatment system may comprise a treatment control system to control one or more parameters of the one or more machines based upon metrics from the set of accelerometers 420 (and/or the set of movement features determined based upon the metrics).

In some examples, the treatment comprises an imaging-based treatment performed using an imaging machine. In some examples, the imaging machine comprises a magnetic resonance imaging (MRI) machine, a computed tomography (CT) scanning machine, an ultrasound machine and/or other type of imaging machine. In some examples, the imaging machine may produce an image (e.g., an MRI scan) at least a portion of the person's body (e.g., the person's head, shoulders, arms, midsection, etc.) positioned within an imaging region of the imaging machine. In an example in which the imaging machine comprises the MRI machine, the MRI machine may produce a magnetic field and/or a radio frequency field in the imaging region, and/or may use the magnetic field and/or the radio frequency field to produce an MRI scan (using MRI sensors of the MRI machine, for example) of a sample in the imaging region (e.g., at least the portion of the person's body within the imaging region). In some examples, the imaging region may correspond to a space defined by an imaging bore of the MRI machine. In some examples, the MRI machine comprises a closed-bore MRI machine comprising a ring of magnets at least partially surrounding the imaging region. Alternatively and/or additionally, the MRI machine may comprise an open MRI machine (e.g., an open-bore MRI machine) comprising a first magnet on a first side of the imaging region and a second magnet on a second side of the imaging region (e.g., the first magnet may be over the imaging region and the second magnet may be under the imaging region). In some examples, the treatment control system may control one or more parameters of the treatment (e.g., one or more parameters of the one or more machines) based upon one or more images (e.g., one or more MRI scans) produced using the imaging machine (e.g., at least one of the MRI machine, the CT scanning machine, the ultrasound machine, etc.) during the treatment.

In some examples, the treatment may comprise a Magnetic Resonance-Guided Focused Ultrasound (MRgFUS) treatment performed using the imaging machine (e.g., at least one of the MRI machine, the CT scanning machine, the ultrasound machine, etc.) and/or an ultrasound device (e.g., a functional ultrasound (fUS) unit). In some examples, the treatment (e.g., the MRgFUS treatment) comprises performing an ablation (e.g., thermoablation) on a target region of the person's body. The target region may correspond a region of the person's brain, such as the brain's thalamus. For example, the target region may correspond to a ventral intermediate nucleus (Vim) of the thalamus. In some examples, the MRgFUS treatment treats one or more diseases (associated with tremors, for example), such as at least one of essential tremor, Parkinson's disease (e.g., tremor-dominant Parkinson's disease), a tumor (e.g., a brain tumor), OCD, epilepsy, neuropathic pain, etc. In an example, the MRgFUS treatment is performed to suppress tremors of the person and/or may lead to improved tremor symptoms (e.g., improved Clinical Rating Scale for Tremor (CRST) scores) of the person.

In some examples, the treatment may comprise emitting, using the ultrasound device, one or more ultrasound energy beams to the target region of the person's body. The target region may be associated with abnormal activity associated with tremors (e.g., the target region may correspond to a source of activity that results in tremors). Heat from the one or more ultrasound energy beams may temporarily increase a temperature of the target region (and/or a lesion may be produced at the target region), which may interrupt the abnormal activity associated with the target region, which may thereby relieve tremors (e.g., future tremors) of the person. In an example, sub-lesional ultrasound energy may from the one or more ultrasound energy beams may temporarily increase the temperature in the thalamus. When the focal temperature increase associated with the sub-lesional ultrasound energy overlaps a cluster of tremor cells, tremor symptoms (e.g., at least one of tremor displacement level, such as the tremor amplitude, tremor frequency, etc.) of the person may be reduced. Increasing the ultrasound energy further may remove and/or destroy (e.g., permanently remove and/or destroy) the tremor cells for sustained benefit. In some examples, the one or more ultrasound energy beams may treat the target region (e.g., remove and/or destroy tremor cells in the target region) without damaging (surrounding) tissue adjacent the target region. During the treatment, the imaging machine may produce one or more images (e.g., at least one of MRI scans, CT scans, ultrasound scans, etc.) of one or more regions of the person (e.g., one or more regions of the person's brain). The one or more images may be used to guide the one or more ultrasound energy beams and/or monitor for changes to one or more regions of the person's body (e.g., at least a portion of the thalamus of the person's brain).

In an example, the one or more images may include images captured at different times. The one or more images may be analyzed to identify impacts of the treatment, such as burning of one or more target regions. The one or more images may be used (in conjunction with the set of movement features and/or the movement assessment report, for example) to determine whether a first target region is sufficiently treated. For example, the one or more images may comprise a first image (e.g., at least one of a first MRI scan, a first CT scan, a first ultrasound scan, etc.) captured at a first time and/or a second image (e.g., at least one of a second MRI scan, a second CT scan, a second ultrasound scan, etc.) captured at a second time. The first image comprises a first representation of the first target region at the first time and/or the second image comprises a second representation of the first target region at the second time. During a period of time between the first time and the second time, the ultrasound device may emit one or more ultrasound energy beams to the first target region to treat (e.g., burn) the first target region. The treatment control system may determine whether the first target region requires further treatment (e.g., further ultrasound energy beams to further heat the first target region) based upon (i) the second image, (ii) a comparison of the first image (before heating the first target region with the one or more ultrasound energy beams) and the second image (after heating the first target region with the one or more ultrasound energy beams), and/or (iii) an improvement in tremor symptoms of the person over the period of time. The improvement in tremor symptoms of the person may be determined based upon a comparison of one or more movement features (e.g., tremor features) derived from metrics collected (using the set of accelerometers 420, for example) prior to the period of time with one or more subsequent movement features (e.g., subsequent tremor features) derived from metrics collected (using the set of accelerometers 420, for example) after the period of time. In some examples, the improvement in tremor symptoms of the person may correspond to a reduction in quantity of tremors per unit of time, a reduction in displacement level of tremors, a reduction in frequency of tremors, a reduction in angular velocity of tremors, etc. In some examples, the improvement in tremor symptoms may be due, at least in part, to the one or more ultrasound energy beams treating the first target region. In some examples, the improvement in tremor symptoms may be an indication that the first target region is sufficiently treated. In some examples, in response to determining that the first target region does not require further ultrasound treatment, the treatment control system may control the ultrasound device to emit one or more further ultrasound energy beams to the first target region to treat (e.g., burn) the first target region. In some examples, in response to determining that the first target region does not require further ultrasound treatment, the treatment control system may control the ultrasound device to (i) cease treating the first target region, (ii) switch from a first state in which the ultrasound device aims at the first target region to a second state in which the ultrasound device aims at a second target region (e.g., a subsequent target region in a sequence of target regions the ultrasound device is configured to treat during the treatment), and/or (iii) emit one or more ultrasound energy beams to the second target region to treat (e.g., burn) the second target region. In some examples, the treatment control system may adjust a target region (treated using the ultrasound device, for example) based upon the set of angles of the one or more joints of the person. For example, based upon a determination that an angle of a joint exceeds a threshold angle (and/or that a tremor severity level associated with the joint exceeds a threshold tremor severity level), the treatment control system may switch the target region to a (predefined, for example) region associated with the joint (e.g., treatment of the region may be known by the treatment control system and/or an observing healthcare professional to impact tremors associated with the joint).

In some examples, the device 406 is operable within the imaging region of the imaging machine (e.g., at least one of the MRI machine, the CT scanning machine, the ultrasound machine, etc.). Alternatively and/or additionally, the device 406 may be operable within a Magnetic Resonance (MR) region of the imaging machine (e.g., the MRI machine). The MR region may correspond to an environment (e.g., at least a portion of a MRI scanning room in which the imaging machine is disposed) affected by the magnetic field produced by the imaging machine (e.g., the MRI machine). In some examples, the device 406 is non-MR interfering, MR compatible, MR safe and/or MR conditional. In some examples, the device 406 is associated with one or more conditions for safe use of the imaging machine. For example, one or more parameters of the imaging machine for the treatment (e.g., the MRgFUS treatment) may be conditions such that the one or more conditions are met. In some examples, the one or more conditions comprise (i) a condition that a static field strength of the imaging machine does not exceed a threshold static field strength, (ii) a condition that a spatial field gradient of the imaging machine does not exceed a threshold spatial field gradient, (iii) a condition that a magnetic field strength of the magnetic field produced by the imaging machine does not exceed a threshold magnetic field strength, and/or (iv) one or more other conditions.

In some examples, the device 406 comprises materials that are selected such that the device 406 is operable within the imaging region and/or the MR region (and/or such that the device 406 is non-MR interfering, MR compatible, MR safe and/or MR conditional). In some examples, manufacturing the device 406 comprises (i) obtaining a first device comprising a first version of the communication device 452, (ii) identifying one or more first components, of the first device, that are (A) not operable within the imaging region, (B) not compatible with the imaging machine (e.g., at least one of the MRI machine, the CT scanning machine, the ultrasound machine, etc.) and/or (C) not safe to operate in the MR region of the imaging machine, and/or (iii) replacing the one or more first components with one or more second components to produce a second version of the communication device 452, wherein the second version of the communication device 452 is operable within the imaging region and/or the MR region (and/or wherein the second version of the communication device 452 is non-MR interfering, MR compatible, MR safe and/or MR conditional).

In some examples, the device 406 comprises the second version of the communication device 452 such that the device 406 is operable within the imaging region and/or the MR region (and/or such that device 406 is non-MR interfering, MR compatible, MR safe and/or MR conditional). In some examples, the one or more first components comprise a first component made of a material (e.g., metal and/or other material) that is at least one of conductive, radiofrequency reactive, magnetic etc. For example, the first component may comprise at least one of a metal screw, a metal switch, a metal case, etc. In some examples, the first component is MR interfering, is not MR compatible, is not MR safe, and/or is MR unsafe, which may be due, at least in part, to a level of magnetic susceptibility of the first component exceeding a threshold level of magnetic susceptibility and/or an electrical conductivity of the first component exceeding a threshold electrical conductivity. The first component may be removed from the communication device 452.

The one or more second components may comprise a second component (e.g., a silicone screw, a silicone switch, a silicone case, etc.) to replace the first component. The second component may be made of a different material than the first component (e.g., the second component may comprise at least one of silicone, rubber, plastic, ceramic, fiberglass, a nonmetallic material, a non-ferromagnetic material, titanium, platinum, aluminum, copper, brass, acrylic, polycarbonate, nickel titanium, etc.). The second component (and/or one or more materials of the second component) may be non-MR interfering, MR compatible and/or MR safe, which may be due, at least in part, to a level of magnetic susceptibility of the second component being less than the threshold level of magnetic susceptibility and/or an electrical conductivity of the second component being less than the threshold electrical conductivity.

Thus, in accordance with some embodiments, the communication device 452 may be manufactured to be operable within the imaging region and/or the MR region (and/or to be non-MR interfering, MR compatible, MR safe and/or MR conditional). In some examples, the set of accelerometers 420 (and/or one or other components of the device 406) and/or the power source 422 (e.g., the battery) may be manufactured and/or designed to be operable within the imaging region and/or the MR region (and/or to be non-MR interfering, MR compatible, MR safe and/or MR conditional), such as using one or more of the techniques provided herein with respect to manufacturing and/or designing the communication device 452. In some examples, the communication device 452 may transmit the message 430 to the first client device 450 via a wireless connection (e.g., Bluetooth connection) or a wired connection that is non-MR interfering.

In some examples, the movement assessment interface may enable the one or more healthcare professionals to observe an impact (e.g., a real-time impact, which may correspond to at least one of an improvement in tremor symptoms, a worsening of tremor symptoms, etc.) of the MRgFUS treatment on the person, which may allow the one or more healthcare professionals (and/or the remote healthcare professional) to tailor one or more aspects of the MRgFUS treatment to the person based upon the observed impact. In some examples, the treatment control system may control the MRgFUS treatment based upon the set of updated treatment parameters (submitted by the one or more healthcare professionals and/or the remote healthcare professional via the treatment control interface, for example). In an example, the set of updated treatment parameters may be indicative of (i) an energy level of one or more ultrasound energy beams emitted by the ultrasound device, (ii) a location of a target region to be treated using the one or more ultrasound energy beams (e.g., thermal ablation using the ultrasound device), (iii) an amount of time to treat the target region (e.g., an amount of time to emit ultrasound energy beams to the target region), (iv) a frequency and/or speed with which images (e.g., at least one of MRI scans, CT scans, ultrasound scans, etc.) are captured by the imaging machine and/or (v) one or more other parameters. In some examples, the treatment control system may control one or more machines associated with the MRgFUS treatment (e.g., the MRI machine and/or the ultrasound device) based upon the set of updated treatment parameters.

In some examples, the treatment guide and/or the treatment control interface may provide guidance, predictions and/or suggestions to modify a parameter (e.g., energy level, target region, etc.) of the treatment (e.g., the MRgFUS treatment) based upon metrics from the set of accelerometers 420 (and/or the set of movement features determined based upon the metrics), such as based upon a determination that one or more first joints have not responded to the treatment as well as one or more second joints. In some examples, a suggestion displayed by the treatment guide and/or the treatment control interface may be indicative of a suggested value of a treatment parameter of the treatment. In an example, the treatment guide and/or the treatment control interface may provide a suggestion to adjust a target region (e.g., switch a current target region to a different target region) in response to determining that a first joint has not responded to the treatment as well as one or more other joints.

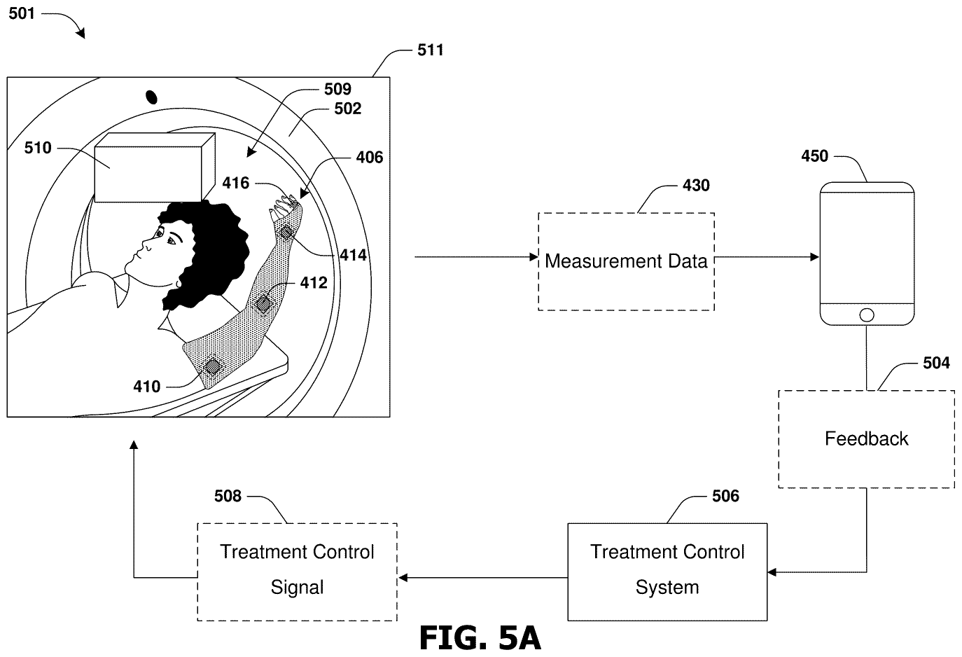

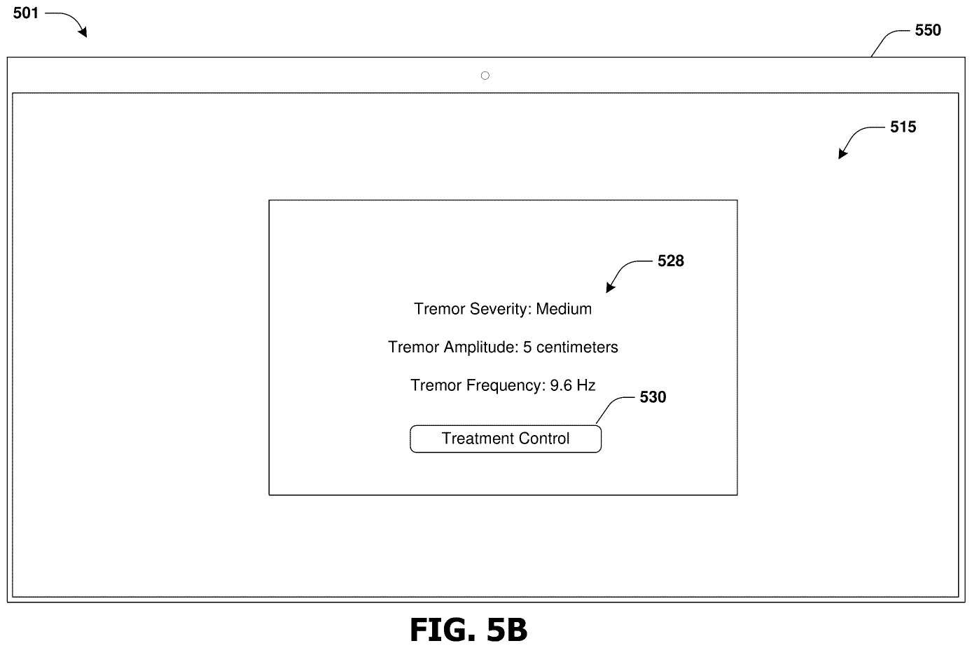

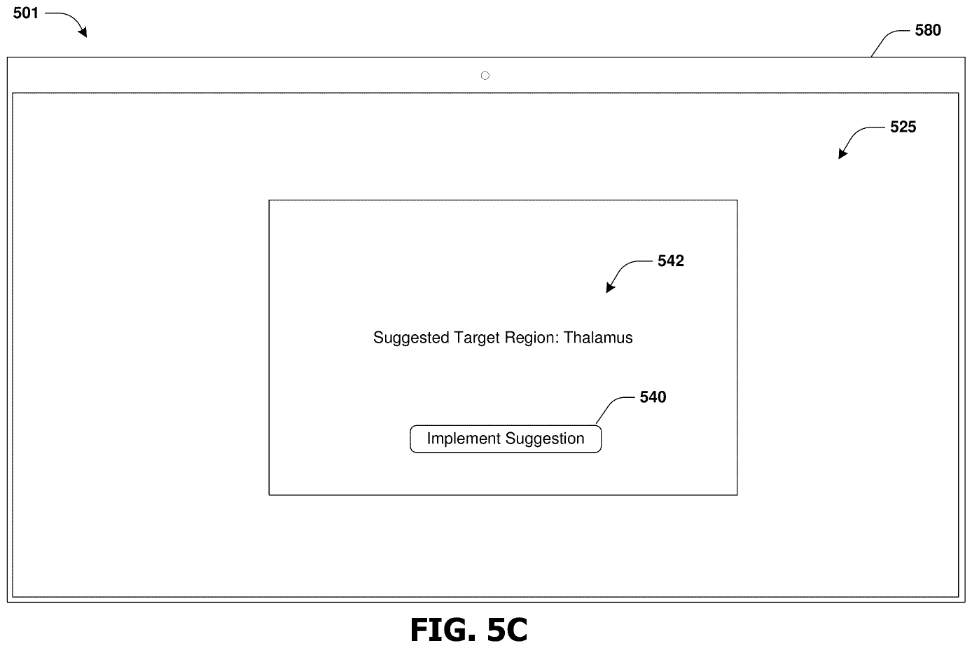

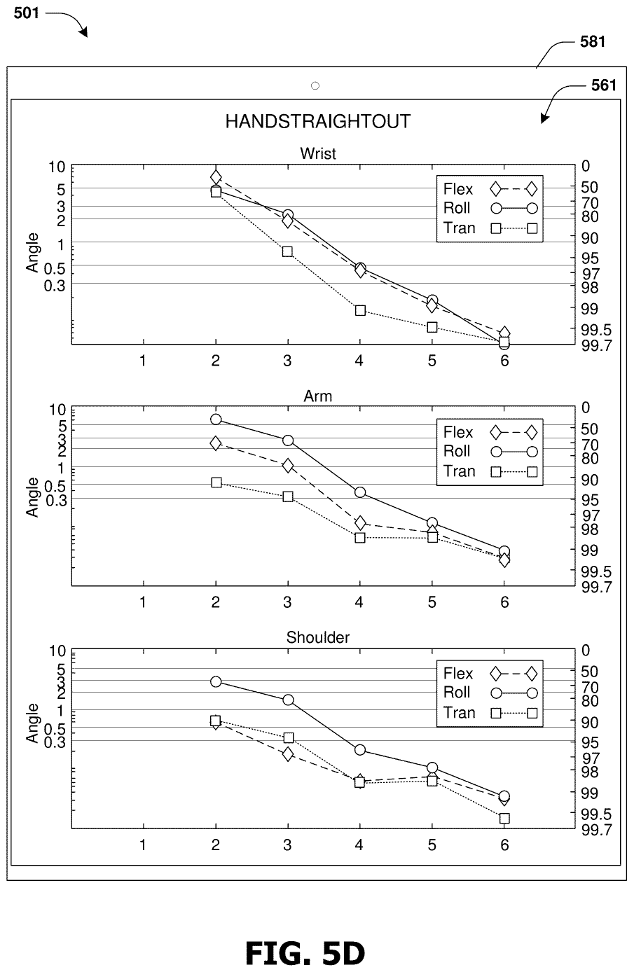

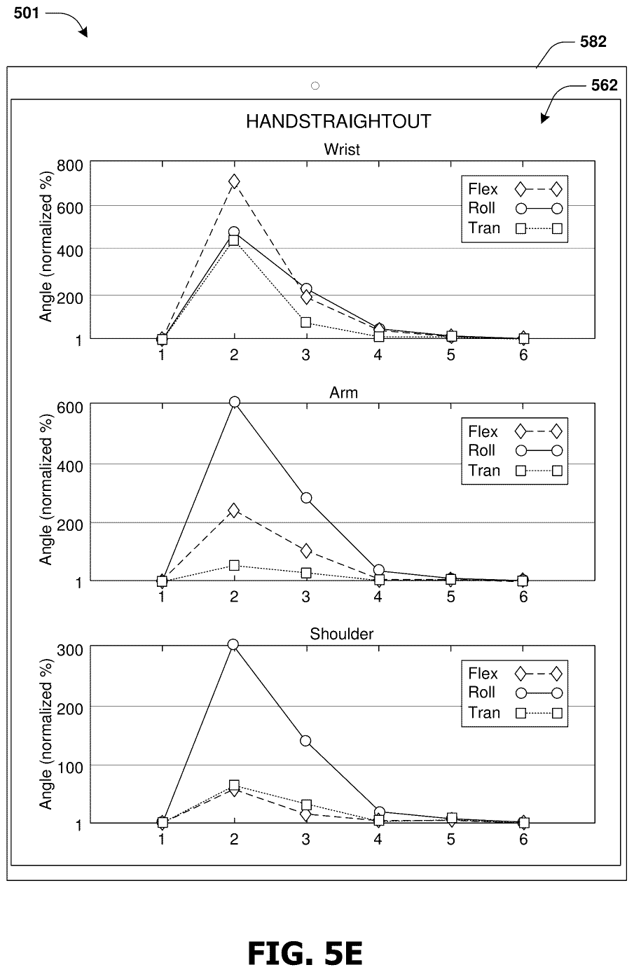

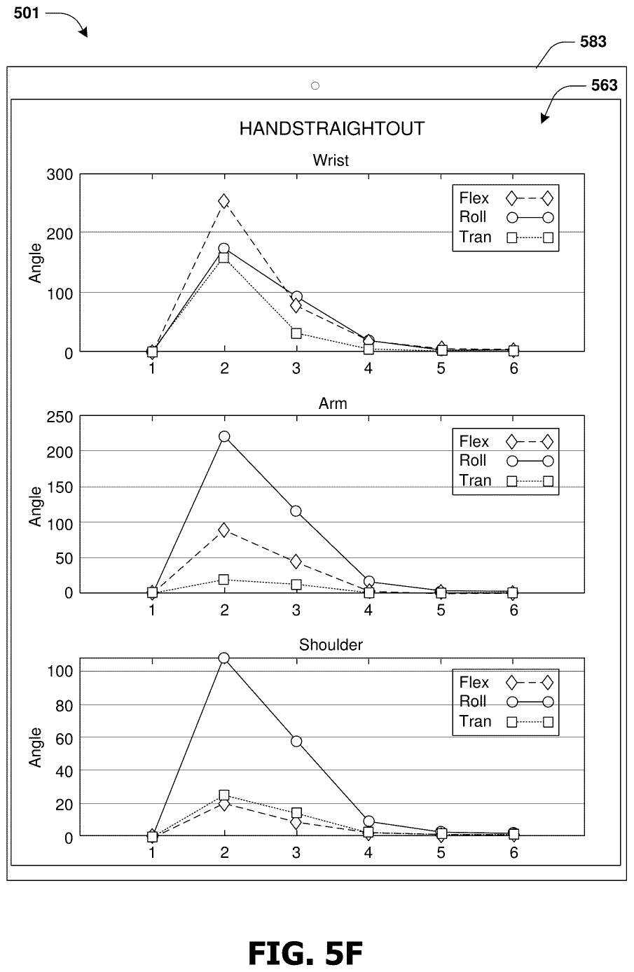

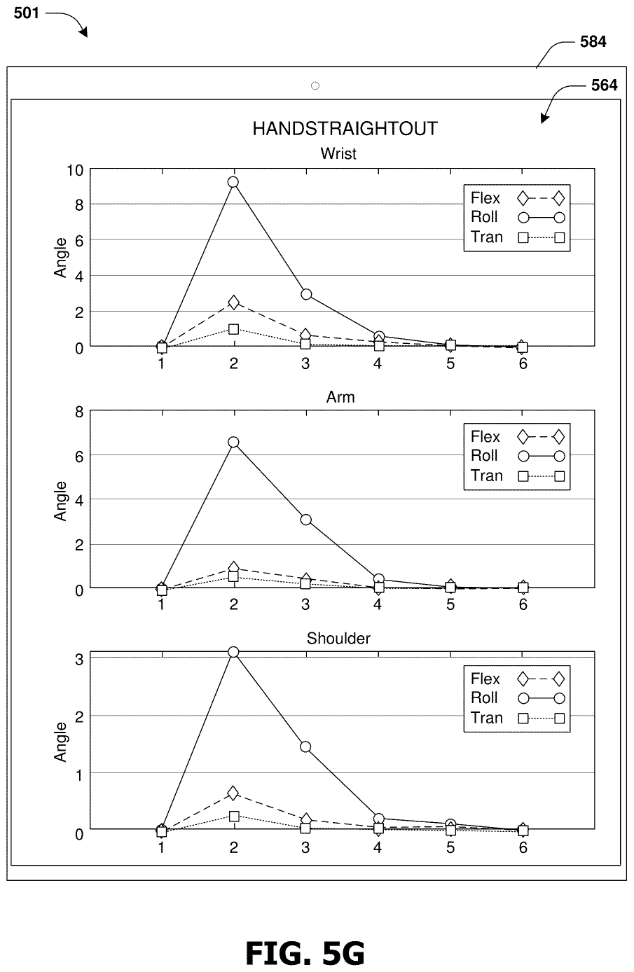

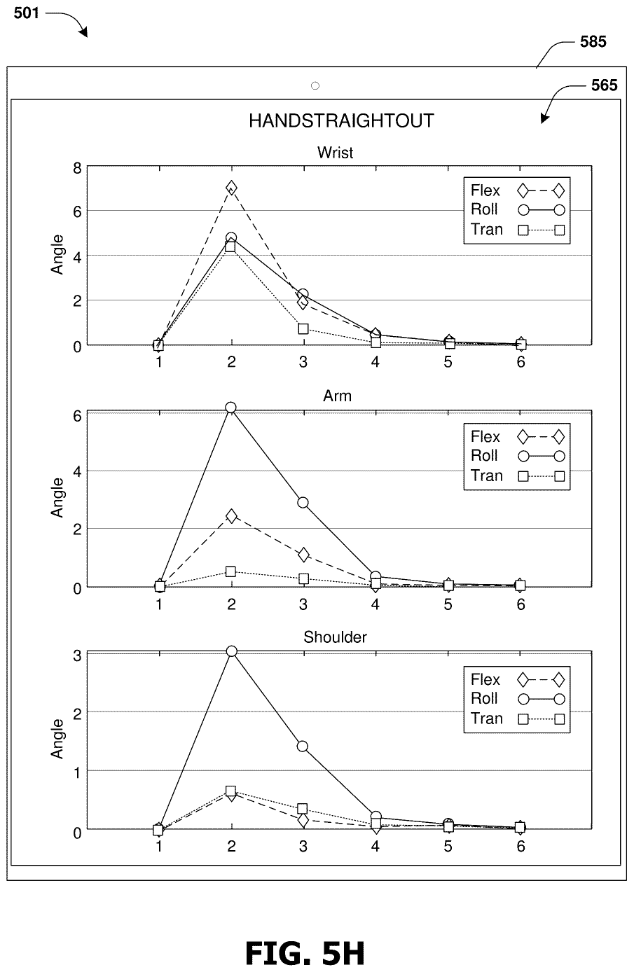

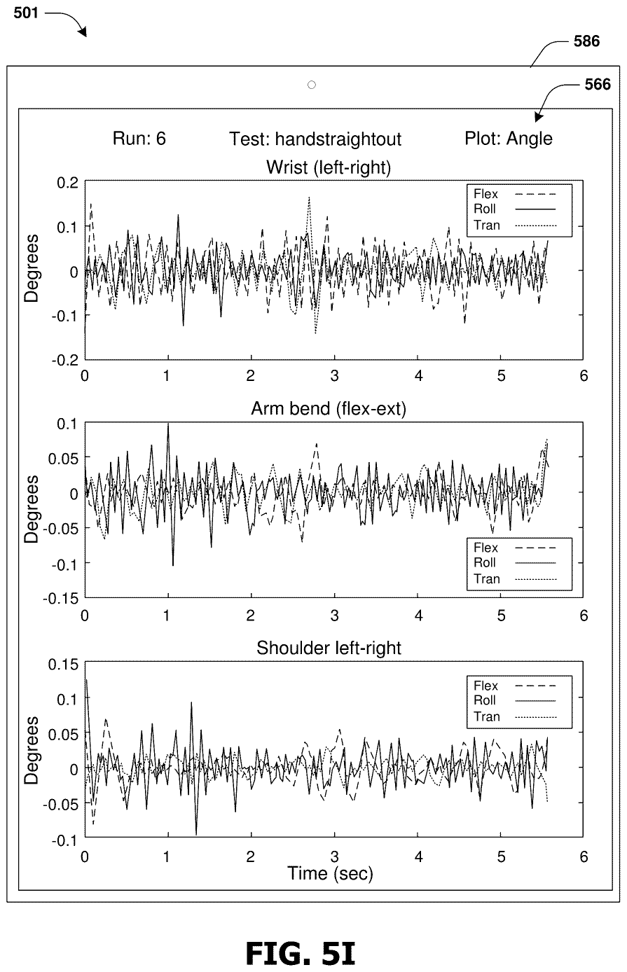

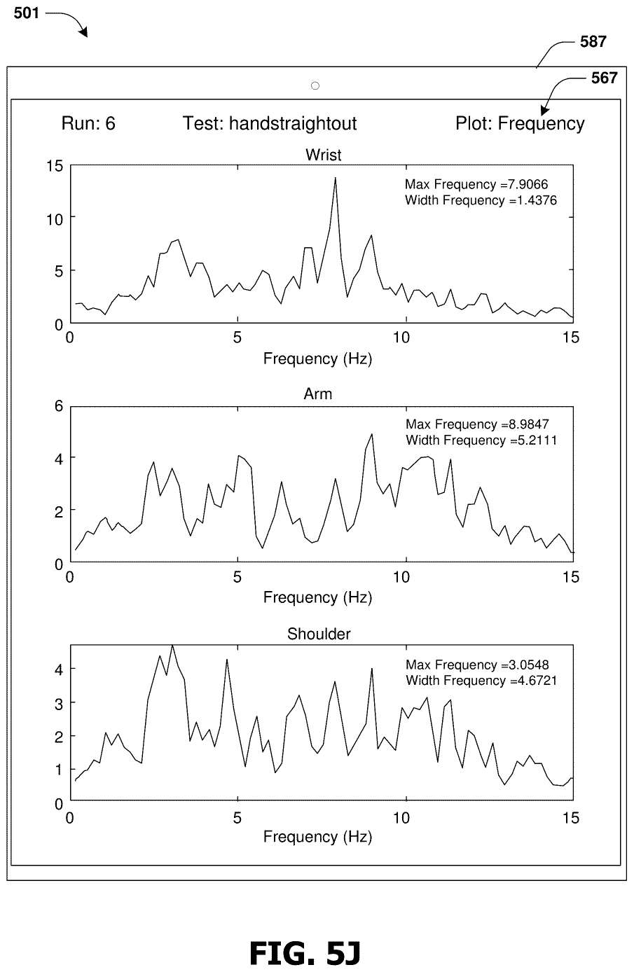

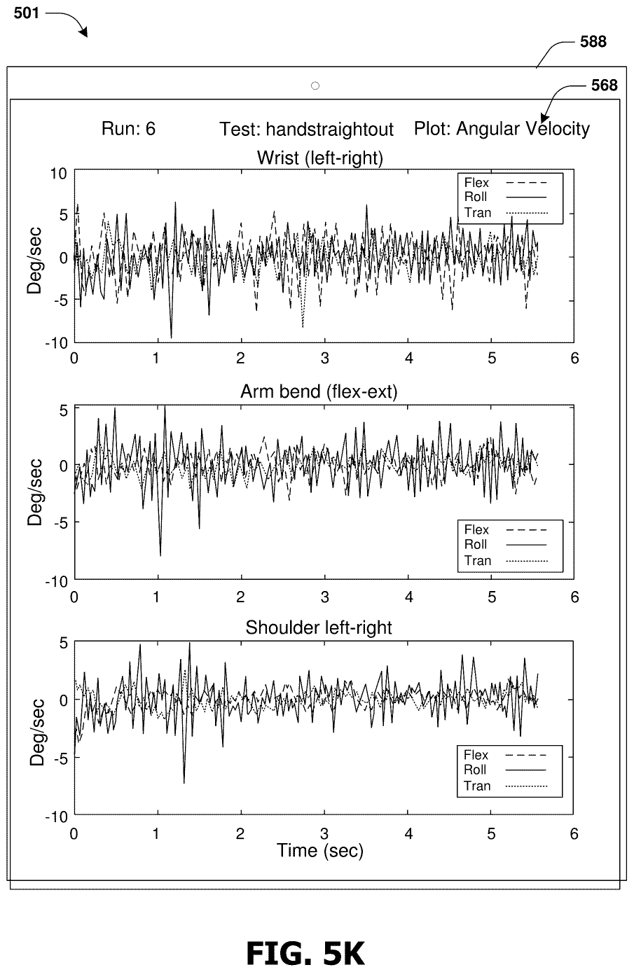

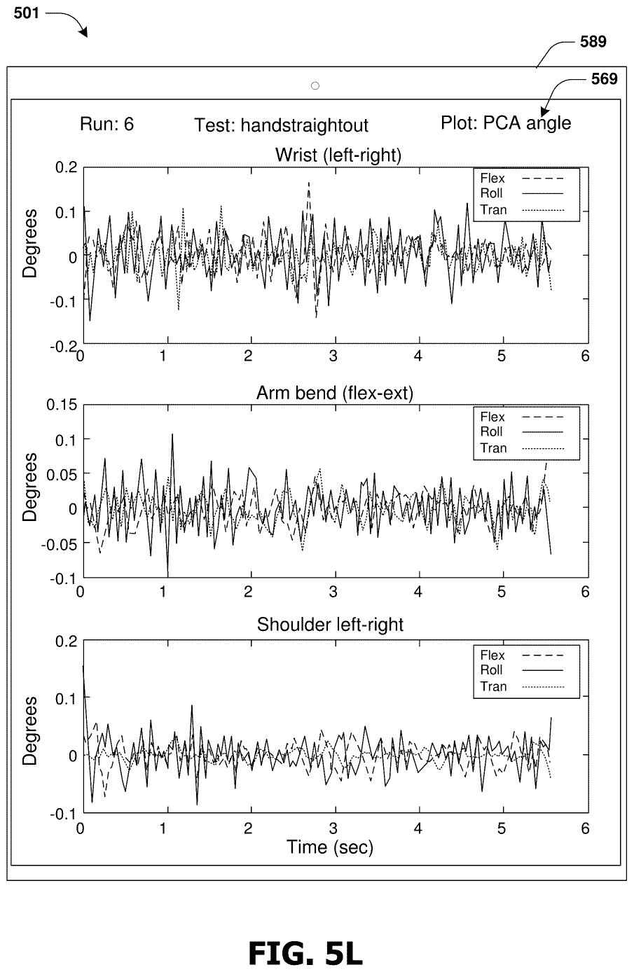

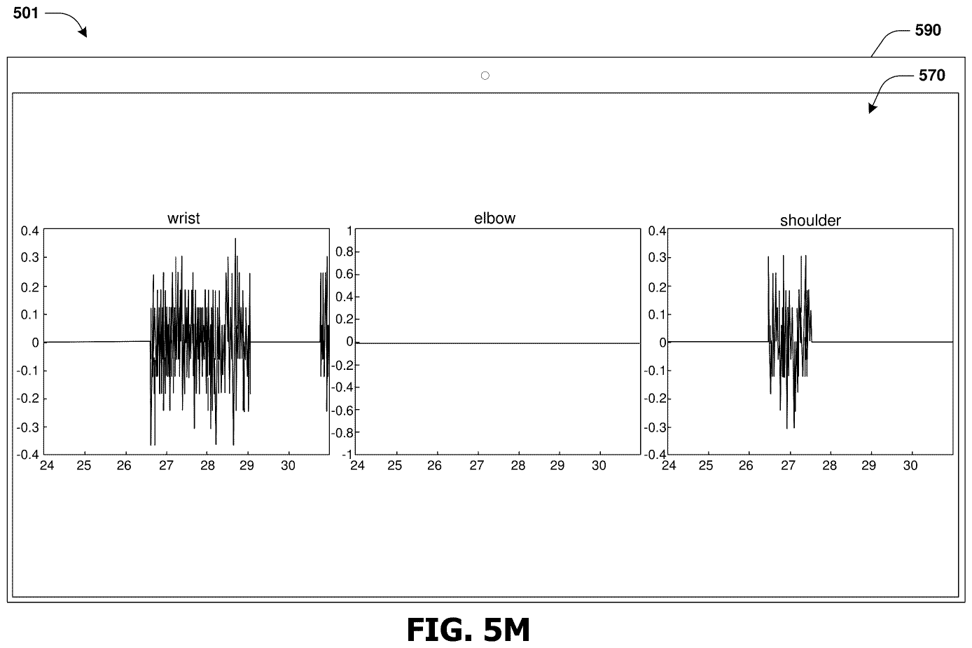

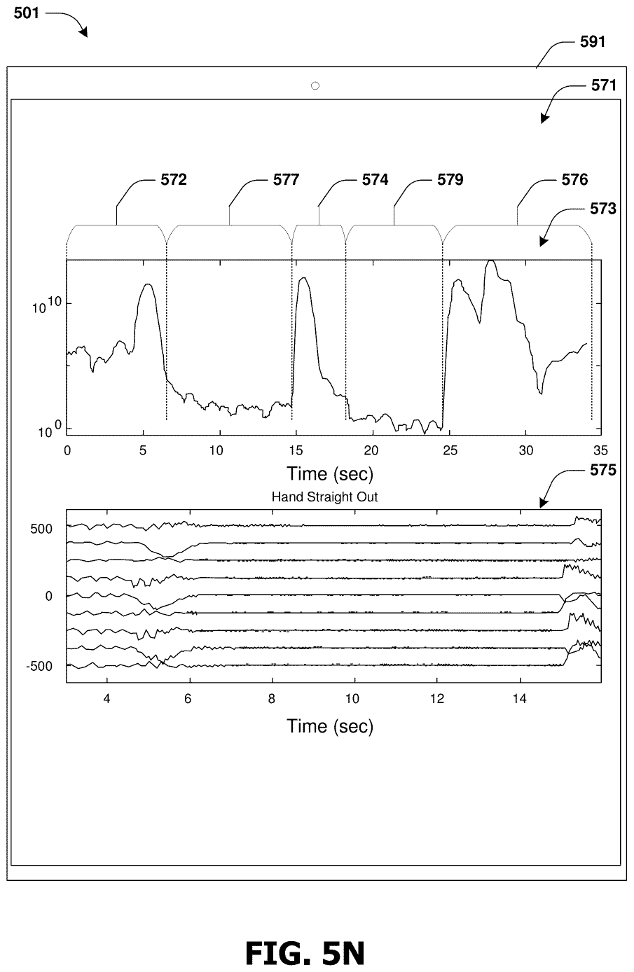

In some examples, the treatment control system may preselect a target region more superiorly in the thalamus on the coronal plane of an MRI scan in response to the metrics from the set of accelerometers 420 (and/or the set of movement features determined based upon the metrics) reporting that the person's tremor is predominantly arising from the person's shoulder. In some examples, the treatment guide and/or the treatment control interface may provide a suggestion (e.g., suggestion 542 in FIG. 5C) to select the (preselected) target region (e.g., the suggestion may be considered by a healthcare professional). In some examples, in response to determining the suggestion, the treatment control system may automatically generate and/or display a selectable input (e.g., selectable input 540 in FIG. 5C) associated with implementing the suggestion. The selectable input may be displayed via the treatment guide and/or the treatment control interface. In some examples, in response to a selection of the selectable input, the treatment control system may perform one or more acts associated with the suggestion. For example, in response to a selection of the selectable input, the treatment control system may instruct one or more machines (e.g., the MRI machine and/or the ultrasound device) to treat the (preselected) target region (by emitting one or more ultrasound energy beams to the target region, for example). Other types of suggestions (displayed by the treatment guide and/or the treatment control interface, for example) are within the scope of the present disclosure.

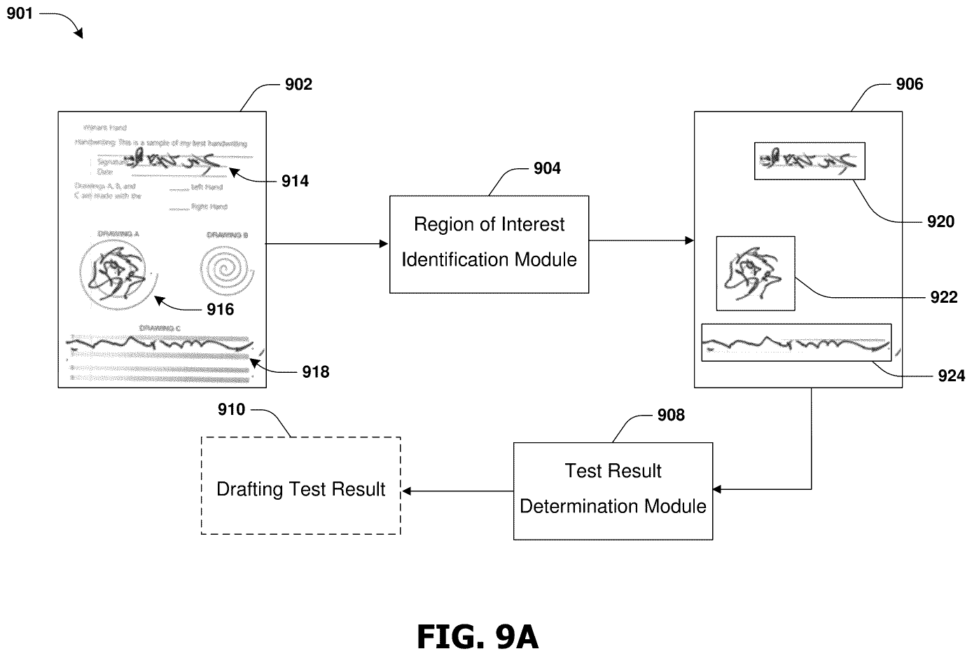

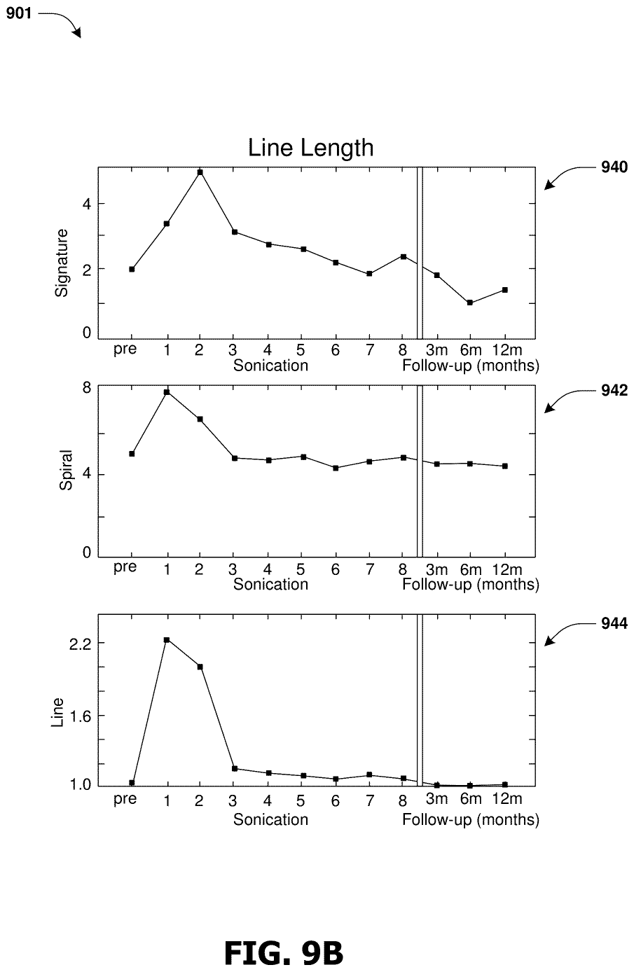

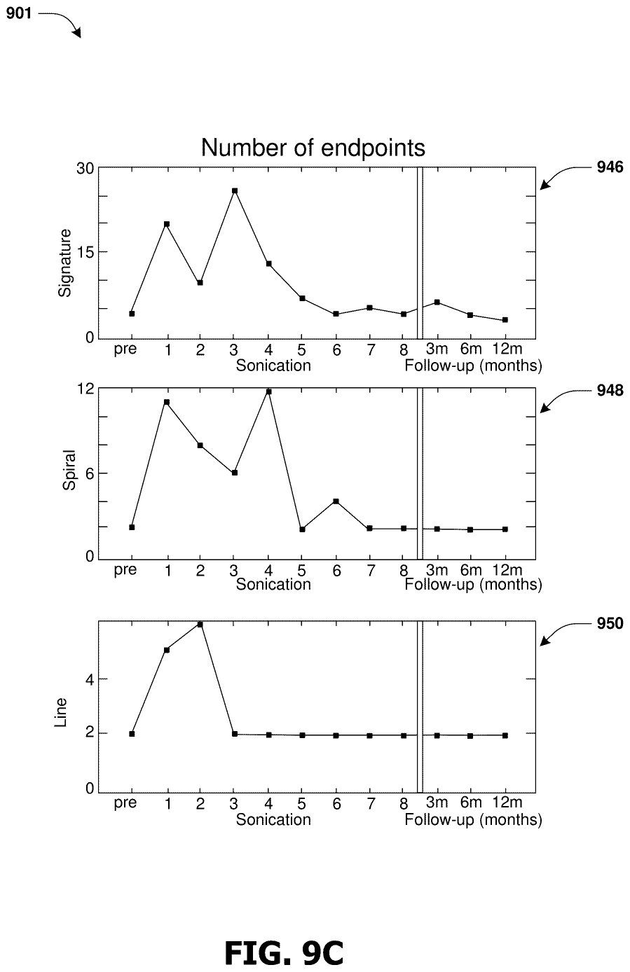

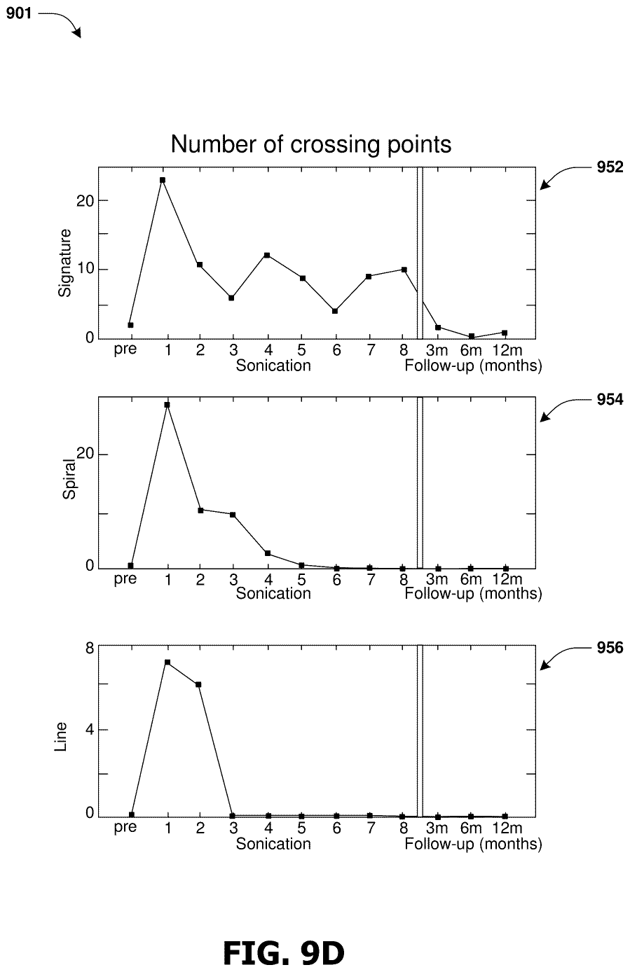

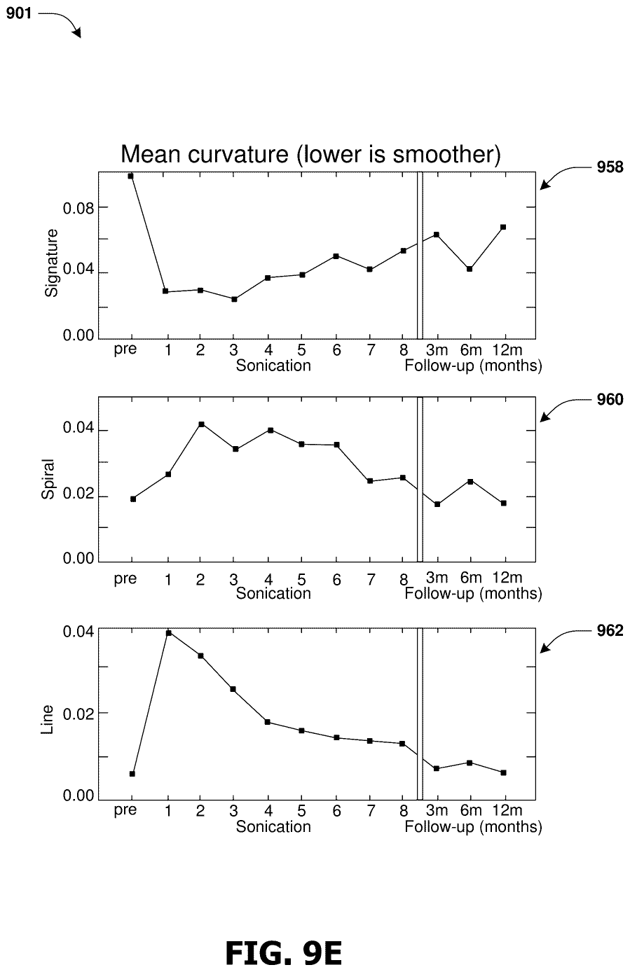

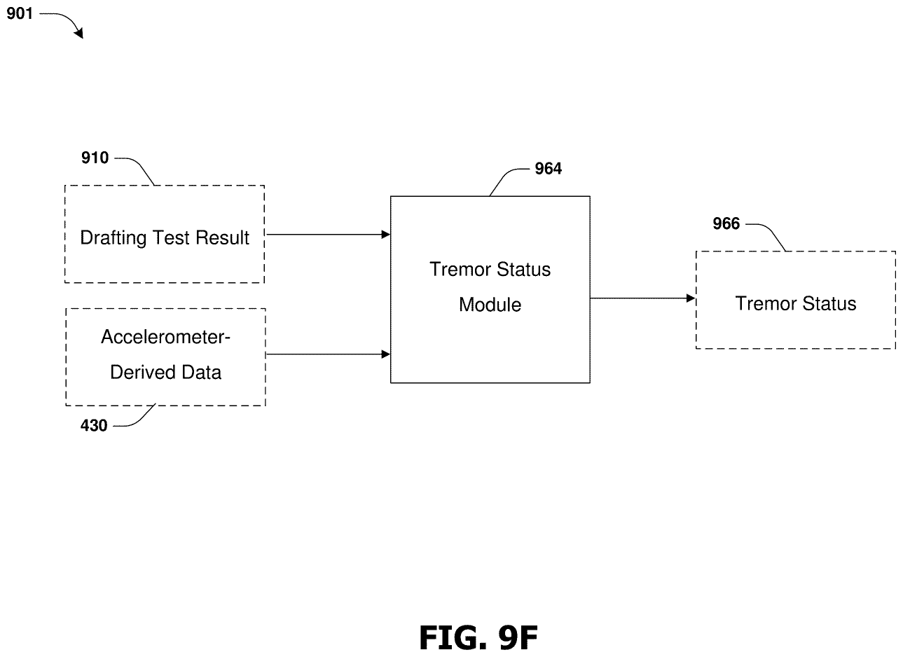

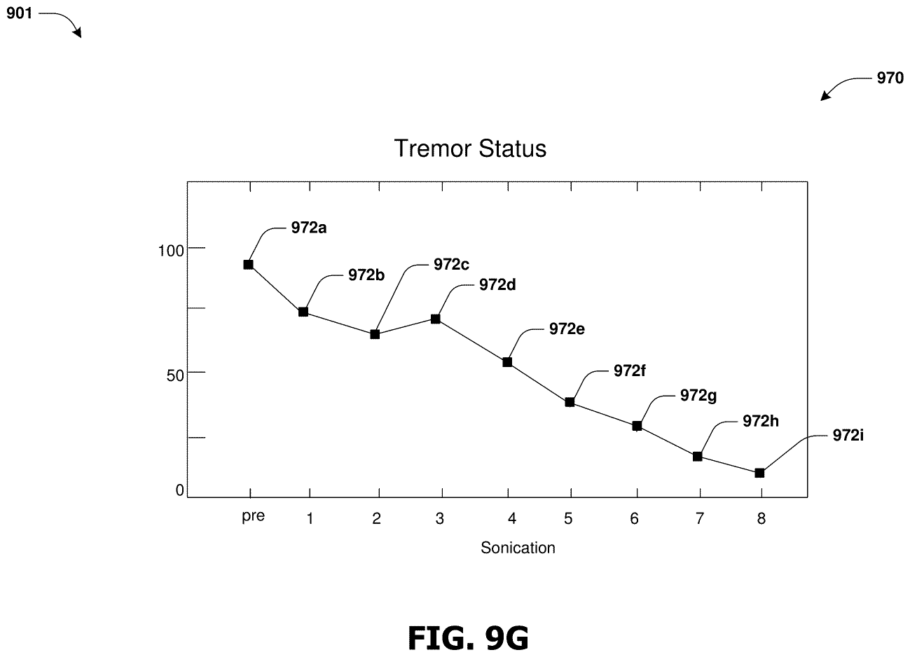

Embodiments are contemplated in which the treatment control system determines the set of updated treatment parameters automatically and/or without manual intervention. In some examples, the set of updated treatment parameters may be determined using a machine learning model. In some examples, the machine learning model may comprise at least one of a tree-based model, a machine learning model used to perform linear regression, a machine learning model used to perform logistic regression, a decision tree model, a support vector machine (SVM), a Bayesian network model, a k-Nearest Neighbors (kNN) model, a K-Means model, a random forest model, a machine learning model used to perform dimensional reduction, a machine learning model used to perform gradient boosting, a neural network model (e.g., a deep neural network model and/or a convolutional neural network model), etc. The machine learning model may be trained using training data to derive updated treatment parameters from an input comprising (i) one or more images (e.g., at least one of MRI scans, CT scans, ultrasound scans, etc.) from the imaging machine, (ii) measurement data from the set of accelerometers 420, (iii) movement features (e.g., the set of movement features) determined based upon the measurement data and/or (iv) drafting test results and/or tremor statuses of the person (determined using one or more of the techniques provided herein with respect to FIGS. 9A-9G, for example). The training data may comprise historical treatment data derived from historical treatments (e.g., MRgFUS treatments). For example, the training data may be indicative of (i) results of the historical treatments, (ii) images (e.g., at least one of MRI scans, CT scans, ultrasound scans, etc.) captured during the historical treatments, (iii) measurement data and/or movement features (determined using accelerometers, for example) associated with the historical treatments, and/or (iv) other training data (e.g., historical drafting tests, historical drafting test results and/or historical tremor statuses determined using one or more of the techniques provided herein with respect to FIGS. 9A-9G).

In some examples, after performing the treatment (e.g., the MRgFUS treatment) of the person, a set of data associated with the treatment may be used to update (e.g., further train) the machine learning model to increase an accuracy of the machine learning model. In an example, the set of data may comprise (i) images captured by the imaging machine for the treatment, (ii) measurement data and/or movement features associated with the person (determined using the device 406), and/or (iii) other information associated with the treatment. In some examples, the machine learning model may be used to determine guidance, predictions and/or suggestions for display on the treatment guide and/or the treatment control interface.

In some examples, the machine learning model may be used to update an ongoing treatment plan (e.g., a treatment plan that the person is current undergoing) to generate an updated version of the ongoing treatment plan and/or to generate a recommended treatment plan (that the person has not yet started, for example) based upon an input comprising (i) one or more images (e.g., at least one of MRI scans, CT scans, ultrasound scans, etc.) from the imaging machine, (ii) measurement data from the set of accelerometers 420 and/or (iii) movement features (e.g., the set of movement features) determined based upon the measurement data. The updated version of the ongoing treatment plan and/or the recommended treatment plan may be indicative of one or more treatments for the person, one or more treatment parameters of the one or more treatments and/or other information. In some examples, the one or more treatments may be controlled based upon the updated version of the ongoing treatment plan and/or the recommended treatment plan. Alternatively and/or additionally, a representation of the updated version of the ongoing treatment plan and/or the recommended treatment plan may be displayed via an interface (e.g., the treatment guide and/or the treatment control interface). Embodiments are contemplated in which a computer program that does not comprise the machine learning model is used (using one or more of the techniques provided herein with respect to the machine learning model, for example) to perform one or more of the acts provided herein with respect to the machine learning model.

In some examples, the machine learning model may be implemented by one or more client devices connected to the remote network. In some examples, a client device connected to the remote network may transmit treatment data determined using the machine learning model (e.g., the set of updated treatment parameters, the updated version of the ongoing treatment plan and/or the recommended treatment plan) to the first client device 450 and/or other client device.

In some examples, the treatment control system may communicate with a console station (e.g., MRgFUS console station comprising the MRI machine and/or the ultrasound device) to extract energy levels and/or heating levels used by the console station in treatments of patients. Historical energy levels and/or heating levels extracted from the console station may be included in the training data to increase an accuracy with which the machine learning model predicts values of parameters of treatments (e.g., MRgFUS treatments). The treatment control system and/or the machine learning model may be updated in response to new training data becoming available (as more patients are treated using the console station, for example) such that the treatment control system and/or the machine learning model continue to learn and/or are reinforced with each patient, for example.

In some examples, the treatment may comprise a deep brain stimulation (DBS) treatment. In some examples, the DBS treatment treats one or more diseases associated with tremors, such as at least one of essential tremor, Parkinson's disease (e.g., tremor-dominant Parkinson's disease), multiple sclerosis, dystonia, etc. In an example, the DBS treatment is performed to suppress tremors of the person and/or may lead to improved tremor symptoms (e.g., improved CRST scores) of the person. Other examples of the treatment other than those explicitly provided herein are within the scope of the present disclosure. In some examples, the treatment may comprise and/or use a brain computer interface.

In some examples, the device 406 may be worn by the person and/or may be used to determine movement features (e.g., tremor features) of the person during one or more periods of time outside of (e.g., prior to and/or after) a period of time during which the treatment is performed. Alternatively and/or additionally, the device 406 may be worn by the person (and/or used to determine movement features of the person) when the person is outside a medical treatment site (e.g., when the person is at home, at a place of work, while commuting, etc.). In some examples, the device 406 and/or the first client device 450 may determine and/or monitor movement features (e.g., tremor features) continuously and/or in a periodic manner (e.g., one or more times per minute, one or more times per hour, one or more times per day, one or more times per week, etc.) and/or an aperiodic manner. In some examples, the movement features may be used to monitor (e.g., keep track of) tremor symptoms (and/or other movement symptoms) of the user (without having to rely on subjective feedback from the person, for example, which may be inaccurate). In some examples, the person may be instructed to war the device 406 and/or activate the device 406 to collect measurement data in a periodic manner (e.g., one or more times per day, one or more times per week, etc.) and/or an aperiodic manner.

In some examples, the device 406, the imaging machine and/or the ultrasound device may be transported (en block, for example) to a delivery location of the person (e.g., the person's home, place of work and/or other location) via a mobile unit (to expedite treatment, for example).

FIG. 5A-5N illustrate a system 501 for facilitating the treatment (e.g., the MRgFUS treatment) of the person. FIG. 5A illustrates a scenario in which the device 406 is used to collect measurement data in association with the treatment (e.g., the MRgFUS treatment) of the person. For example, the device 406 may collect measurements during the treatment, which may be enabled, at least in part, by the device 406 being operable within the imaging region (e.g., imaging bore 509 in FIG. 5A) and/or the MR region (shown with reference number 511). The device 406 may transmit the message 430 indicative of the measurement data (collected using the set of accelerometers 420 of the device 406, for example) to the first client device 450. In some examples, the first client device 450 may be located in a MR console room (adjacent the MRI scanning room, for example) and/or may be configured to record and/or parse incoming measurement data from the device 406. In some examples, during the treatment, the device 406 (e.g., the communication device 452 of the device 406) may transmit measurement data to the first client device 450 in at least one of a continuous manner, a periodic manner, and/or an aperiodic manner. The message 430 (and/or other messages indicative of measurement data collected by the device 406) may be usable to control one or more machines used to perform the treatment, such as the MRI machine (shown with reference number 502) and/or the ultrasound device (shown with reference number 510). In some examples, the message 430 (and/or other messages indicative of measurement data collected by the device 406) may be transmitted while the one or more machines are used to perform the treatment (e.g., the MRgFUS treatment) of the person.

In some examples, the first client device 450 may process the message 530 to generate treatment response feedback 504 indicative of at least some of the measurement data. In some examples, the treatment response feedback 504 may be indicative of the set of movement features (e.g., the set of tremor features) associated with the person. The treatment response feedback 504 may be transmitted to the treatment control system (shown with reference number 506). The treatment control system 506 may provide a treatment control signal 508 based upon the treatment response feedback 504. For example, the treatment control signal 508 may be indicative of a set of updated treatment parameters. The treatment control signal 508 may be transmitted to one, some and/or all of the one or more machines (e.g., the MRI machine 502 and/or the ultrasound device 510) used to perform the treatment. In an example, the set of updated treatment parameters (indicated by the treatment control signal 508) may comprise one or more parameters associated with the ultrasound device 510 comprising (i) a first amount of time to treat a first target region, (ii) a first location of the first target region, (iii) a first energy level associated with treating the first target region, and/or (iv) one or more other parameters. In response to the treatment control signal 508, the ultrasound device 510 may set a current target region to the first location, and/or may emit one or more ultrasound energy beams according to the first energy level to the current target region (e.g., the first target region) for the first amount of time. Alternatively and/or additionally, the set of updated treatment parameters (indicated by the treatment control signal 508) may comprise one or more parameters associated with the MRI machine 502 comprising a frequency and/or speed with which to capture images (e.g., at least one of MRI scans, CT scans, ultrasound scans, etc.) and/or one or more other parameters.

In some examples, the treatment control signal 508 (and/or the set of updated treatment parameters) may be determined based upon information submitted (e.g., manually submitted by a healthcare professional) using the treatment control interface, which may be displayed to an on-site healthcare professional (via a display in the MR console room, for example) and/or to a remote healthcare professional (via the remote client device, for example). In some examples, the treatment control interface and/or the movement assessment interface may be displayed concurrently (e.g., concurrently on a single display and/or concurrently on separate displays) to the on-site healthcare professional and/or to the remote healthcare professional. Alternatively and/or additionally, the treatment control signal 508 (and/or the set of updated treatment parameters) may be determined using the machine learning model.

FIG. 5B illustrates a client device 550 (e.g., the remote client device and/or an on-site client device) displaying a portion 515 of the movement assessment interface. In some examples, the portion 515 of the movement assessment interface may display a set of information 528 comprising an indication of a tremor severity level associated with one or more detected tremors of the person, an indication of a tremor displacement level (e.g., a tremor amplitude) of the one or more detected tremors, an indication of a tremor frequency of the one or more detected tremors and/or an indication of an angular velocity of the one or more detected tremors. In some examples, the portion 515 of the movement assessment interface may display a selectable input 530 associated with accessing the treatment control interface. In some examples, the treatment control interface may be displayed in response to a selection of the selectable input 530.

FIG. 5C illustrates a client device 580 (e.g., the remote client device and/or an on-site client device) displaying the treatment control interface (shown with reference number 525). In some examples, the treatment control interface 525 may display the suggestion 542 to select a target region (e.g., thalamus) and/or the selectable input 540 associated with implementing the suggestion 542.

FIG. 5D illustrates a client device 581 (e.g., the remote client device and/or an on-site client device) displaying a portion 561 of the movement assessment interface. The portion 561 of the movement assessment interface may comprise (i) a representation of a movement feature corresponding to an angle associated with the person's wrist over time, (ii) a representation of a movement feature corresponding to an angle associated with the person's arm over time, and/or (iii) a representation of a movement feature corresponding to an angle associated with the person's shoulder over time. FIG. 5E illustrates a client device 582 (e.g., the remote client device and/or an on-site client device) displaying a portion 562 of the movement assessment interface. The portion 562 of the movement assessment interface may comprise (i) a representation of a movement feature corresponding to a normalized angle associated with the person's wrist over time, (ii) a representation of a movement feature corresponding to a normalized angle associated with the person's arm over time, and/or (iii) a representation of a movement feature corresponding to a normalized angle associated with the person's shoulder over time. FIG. 5F illustrates a client device 583 (e.g., the remote client device and/or an on-site client device) displaying a portion 563 of the movement assessment interface. The portion 563 of the movement assessment interface may comprise (i) a representation of a movement feature corresponding to an angle associated with the person's wrist over time, (ii) a representation of a movement feature corresponding to an angle associated with the person's arm over time, and/or (iii) a representation of a movement feature corresponding to an angle associated with the person's shoulder over time. FIG. 5G illustrates a client device 584 (e.g., the remote client device and/or an on-site client device) displaying a portion 564 of the movement assessment interface. The portion 564 of the movement assessment interface may comprise (i) a representation of a movement feature corresponding to an angle associated with the person's wrist over time, (ii) a representation of a movement feature corresponding to an angle associated with the person's arm over time, and/or (iii) a representation of a movement feature corresponding to an angle associated with the person's shoulder over time. FIG. 5H illustrates a client device 585 (e.g., the remote client device and/or an on-site client device) displaying a portion 565 of the movement assessment interface. The portion 565 of the movement assessment interface may comprise (i) a representation of a movement feature corresponding to an angle associated with the person's wrist over time, (ii) a representation of a movement feature corresponding to an angle associated with the person's arm over time, and/or (iii) a representation of a movement feature corresponding to an angle associated with the person's shoulder over time. FIG. 5I illustrates a client device 586 (e.g., the remote client device and/or an on-site client device) displaying a portion 566 of the movement assessment interface. The portion 566 of the movement assessment interface may comprise (i) a representation of a movement feature corresponding to an angle associated with the person's wrist over time, (ii) a representation of a movement feature corresponding to an angle associated with the person's arm over time, and/or (iii) a representation of a movement feature corresponding to an angle associated with the person's shoulder over time. FIG. 5J illustrates a client device 587 (e.g., the remote client device and/or an on-site client device) displaying a portion 567 of the movement assessment interface. The portion 567 of the movement assessment interface may comprise (i) a representation of a movement feature corresponding to a frequency associated with the person's wrist, (ii) a representation of a movement feature corresponding to a frequency associated with the person's arm, and/or (iii) a representation of a movement feature corresponding to a frequency associated with the person's shoulder. FIG. 5K illustrates a client device 588 (e.g., the remote client device and/or an on-site client device) displaying a portion 568 of the movement assessment interface. The portion 568 of the movement assessment interface may comprise (i) a representation of a movement feature corresponding to an angular velocity associated with the person's wrist over time, (ii) a representation of a movement feature corresponding to an angular velocity associated with the person's arm over time, and/or (iii) a representation of a movement feature corresponding to an angular velocity associated with the person's shoulder over time. FIG. 5L illustrates a client device 589 (e.g., the remote client device and/or an on-site client device) displaying a portion 569 of the movement assessment interface. The portion 569 of the movement assessment interface may comprise (i) a representation of a movement feature corresponding to a principal component analysis (PCA) angle associated with the person's wrist over time, (ii) a representation of a movement feature corresponding to a PCA angle associated with the person's arm over time, and/or (iii) a representation of a movement feature corresponding to a PCA angle associated with the person's shoulder over time. FIG. 5M illustrates a client device 590 (e.g., the remote client device and/or an on-site client device) displaying a portion 570 of the movement assessment interface. The portion 570 of the movement assessment interface may comprise (i) a representation of a movement feature associated with the person's wrist over time, (ii) a representation of a movement feature associated with the person's elbow over time, and/or (iii) a representation of a movement feature associated with the person's shoulder over time. FIG. 5N illustrates a client device 591 (e.g., the remote client device and/or an on-site client device) displaying a portion 571 of the movement assessment interface. The portion 571 of the movement assessment interface may comprise (i) a representation 573 of a movement feature associated with the person over time (e.g., the representation 573 may comprise a curve corresponding to a combination, such as an average, of values output by at least some accelerometers of the set of accelerometers), and/or (ii) a representation 575 of movement features associated with the person over time (e.g., each curve of the representation 575 may correspond to values output by an accelerometer of the set of accelerometers). In some examples, the representation 573 and/or the representation 575 may be a representation of at least some measurement data indicated by the message 430. In some examples, the treatment system (and/or the treatment control system) may identify (i) one or more first time periods (e.g., time periods 577 and/or 579) in which the person is determined to have a testing position and/or (ii) one or more second time periods (e.g., time periods 572, 574 and/or 576) in which the person is determined not to have the testing position. For example, the treatment system (and/or the treatment control system) may identify the one or more first time periods and/or the one or more second time periods by analyzing measurement data indicated by the message 430. In an example, the testing position may correspond to the person's hand and/or arm being extended in a straight manner. First data output by the set of accelerometers when the person is in the testing position may be usable for evaluating tremors of the person. In some examples, second data output by the set of accelerometers when the person is not in the testing position may not be usable for (and/or may be of relatively worse quality compared with the first data) for evaluating tremors of the person, such as due to the second data including movement information for non-tremor movements. In some examples, the treatment system (and/or the treatment control system) may use the first data (and/or other data associated with the one or more first time periods) to determine the set of movement features (e.g., the set of tremor features), the tremor severity level of the person, the treatment control signal 508 and/or the set of updated treatment parameters. Alternatively and/or additionally, the treatment system (and/or the treatment control system) may filter the second data (and/or other data associated with the one or more second time periods) from data that is used to determine the set of movement features (e.g., the set of tremor features), the tremor severity level of the person, the treatment control signal 508 and/or the set of updated treatment parameters.

Embodiments are contemplated in which the portions 513, 515, 561, 562, 563, 564, 565, 566, 567, 568, 569, 570 and/or 571 of the movement assessment interface are displayed (e.g., concurrently displayed) on the same display. In some examples, one, some and/or all of the operations of in the present disclosure that are described as being performed by the first client device 450 may be performed by one or more other entities, such as at least one of the treatment system, the treatment control system 506, a server, the remote client device, one or more other client devices, etc.

In some examples, the device 406 may comprise an indicator device configured to output a treatment indication based upon the measurement data. In some examples, the treatment indication is usable to control a treatment (e.g., a radiological treatment and/or other type of treatment) of the person. Alternatively and/or additionally, the treatment indication may be indicative of one or more movement features (e.g., one or more tremor features). The one or more movement features may comprise a tremor severity level associated with one or more detected tremors of the person, a tremor displacement level (e.g., a tremor amplitude) of the one or more detected tremors, a tremor angular velocity of the one or more detected tremors, a tremor angle of the one or more detected tremors and/or a tremor frequency of the one or more detected tremors. In some examples, information indicated by the indictor device (e.g., at least one of the treatment indication, the one or more movement features, etc.) may be determined using a controller of the device 406 and/or using one or more accelerometers of the set of accelerometers.

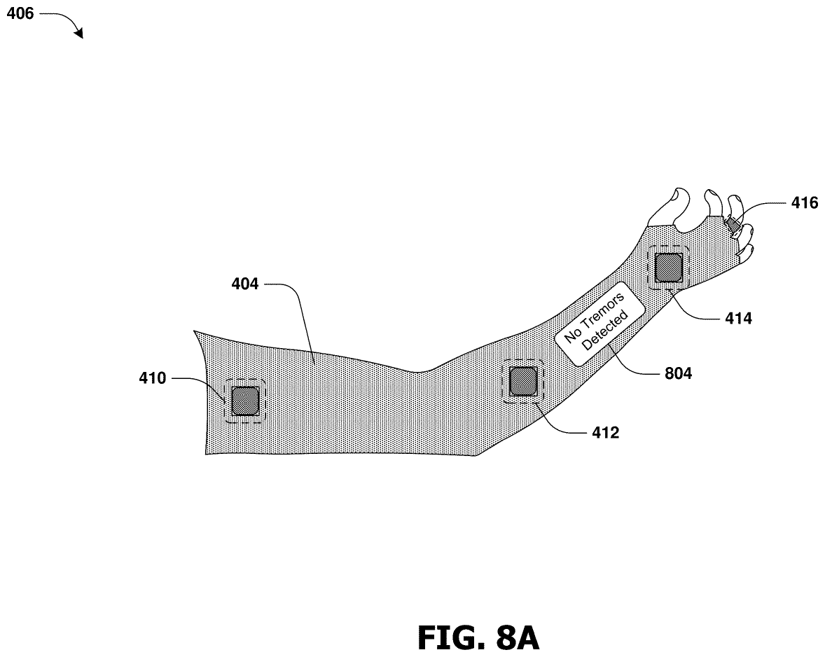

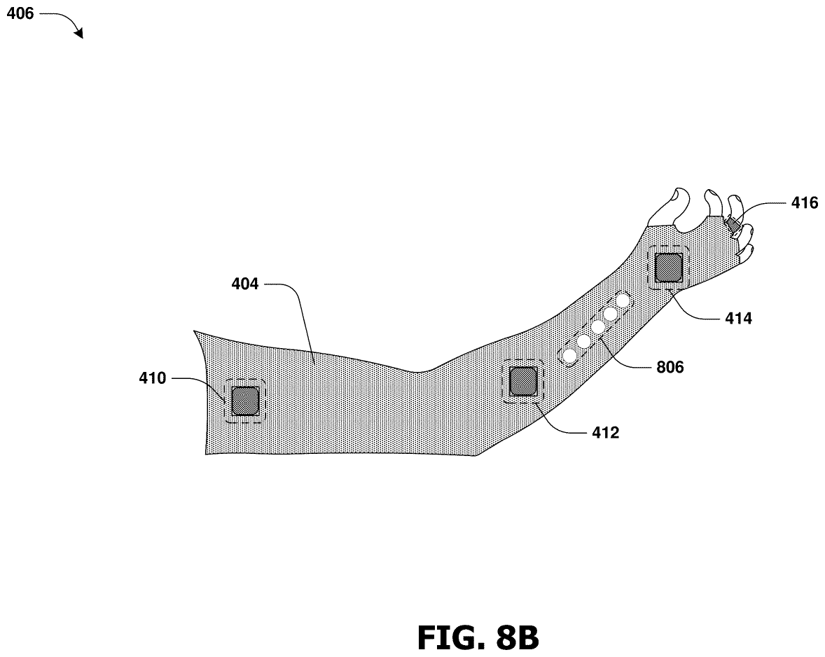

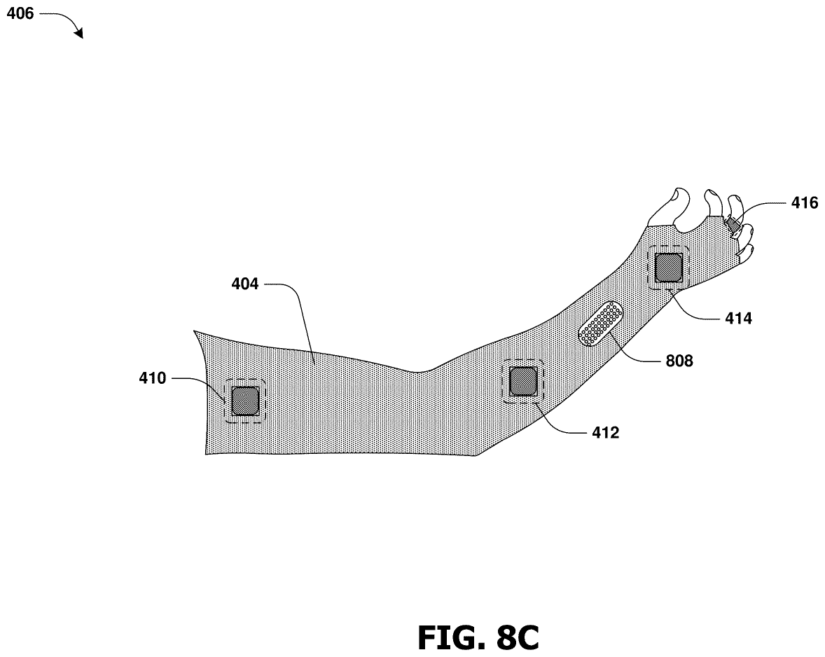

In some examples, the indicator device comprises a display 804 configured to produce a visual indication of the treatment indication and/or one or more movement features. FIG. 8A illustrates the device 406 comprising the display 804, according to some embodiments. The display 804 may comprise a seven-segment display, a dot matrix display, a liquid crystal display (LCD), a touchscreen and/or other type of display. The display 804 may be attached to the sleeve 404 and/or at least partially embedded in the sleeve 404 (e.g., embedded in a material of the sleeve 404). In some examples, the display 804 may display a graphical object (e.g., at least one of a set of text, an image, an icon, etc.) indicative of one or more movement features (e.g., the tremor severity level, the tremor displacement level, the tremor angular velocity, the tremor angle and/or the tremor frequency). Alternatively and/or additionally, the display 804 may display a graphical object (e.g., at least one of a set of text, an image, an icon, etc.) indicative of guidance, predictions and/or suggestions to modify a parameter (e.g., energy level, target region, etc.) of the treatment. For example, the graphical object may be indicative of at least one of a suggested value of a treatment parameter of the treatment, a suggestion to adjust a target region (e.g., switch a current target region to a different target region), etc.

In some examples, the indicator device comprises a set of light sources 806 (e.g., a set of one or more light sources) configured to produce a visual indication of the treatment indication and/or one or more movement features. In some examples, a light source of the set of light sources 806 comprises a light-emitting diode (LED) and/or other type of light source. FIG. 8B illustrates the device 406 comprising the set of light sources 806, according to some embodiments. The set of light sources 806 may be attached to the sleeve 404 and/or at least partially embedded in the sleeve 404 (e.g., embedded in a material of the sleeve 404). In some examples, colors, intensities and/or patterns of light emitted by the set of light sources 806 may be indicative of one or more movement features (e.g., the tremor severity level, the tremor displacement level, the tremor angular velocity, the tremor angle and/or the tremor frequency). For example, the one or more movement features may be indicated by a quantity of light sources of the set of light sources 806 that are activated to emit light (e.g., a greater quantity of activated light sources may indicate a greater tremor severity level). Alternatively and/or additionally, the one or more movement features may be indicated by a color of light emitted by a light source of the set of light sources 806 that are activated to emit light (e.g., the light source emitting a red light may indicate a greater tremor severity level than the light source emitting a green light). Alternatively and/or additionally, the one or more movement features may be indicated by an intensity of light emitted by a light source of the set of light sources 806 that are activated to emit light (e.g., a greater intensity of light emitted by the light source may indicate a greater tremor severity level). Alternatively and/or additionally, colors, intensities and/or patterns of light emitted by the set of light sources 806 may be indicative of indicative of guidance, predictions and/or suggestions to modify a parameter (e.g., energy level, target region, etc.) of the treatment.

In some examples, the indicator device comprises a speaker 808 configured to produce audio corresponding to the treatment indication and/or the one or more movement features. FIG. 8C illustrates the device 406 comprising the speaker 808, according to some embodiments. The speaker 808 may be attached to the sleeve 404 and/or at least partially embedded in the sleeve 404 (e.g., embedded in a material of the sleeve 404). In some examples, audio emitted by the speaker 808 may be indicative of one or more movement features (e.g., the tremor severity level, the tremor displacement level, the tremor angular velocity, the tremor angle and/or the tremor frequency). For example, the one or more movement features may be indicated by a tone of the audio (e.g., a tone with a greater frequency may indicate a greater tremor severity level). Alternatively and/or additionally, the one or more movement features may be indicated by speech of the audio. Alternatively and/or additionally, the audio (comprising speech, for example) emitted by the speaker 808 may be indicative of guidance, predictions and/or suggestions to modify a parameter (e.g., energy level, target region, etc.) of the treatment. For example, the audio may comprise speech comprising “increase ultrasound beam energy level”. Alternatively and/or additionally, the audio (comprising speech, for example) emitted by the speaker 808 may be indicative of one or more instructions for the person. For example, the audio may comprise speech comprising “move your arm upwards”. Embodiments are contemplated in which the speaker 808 is external to the device 406 (and/or where the speaker 808 is a speaker of the first client device 450 or other device).

In some examples, a side effect of the treatment may be a loss and/or reduction in sensation. In some examples, the device 406 may comprise a sensation checker (not shown). The sensation checker may be attached to the sleeve 404 and/or at least partially embedded in the sleeve 404 (e.g., embedded in a material of the sleeve 404). When activated, the sensation checker may at least one of pulsate, vibrate, move, apply a force (to the person's hand, for example), etc. The person may be asked whether they feel at least one of the pulsation, vibration, movement, applied force, etc. of the sensation checker. The person's response may be used to (i) determine whether the person experienced a loss and/or reduction in sensation and/or (ii) control the treatment. In some examples, the sensation checker is positioned proximal the person's hand (e.g., a finger of the hand) and/or other body part of the person. In some examples, the sensation checker is controlled (e.g., activated and/or deactivated) using at least one of the first client device 450, the treatment control system 506, a server, the remote client device, one or more other client devices, etc.

In some examples, one, some and/or all of the operations of in the present disclosure that are described as being performed by the treatment system and/or the treatment control system 506 may be performed by one or more other entities, such as at least one of the first client device 450, the treatment control system 506, a server, the remote client device, one or more other client devices, etc.

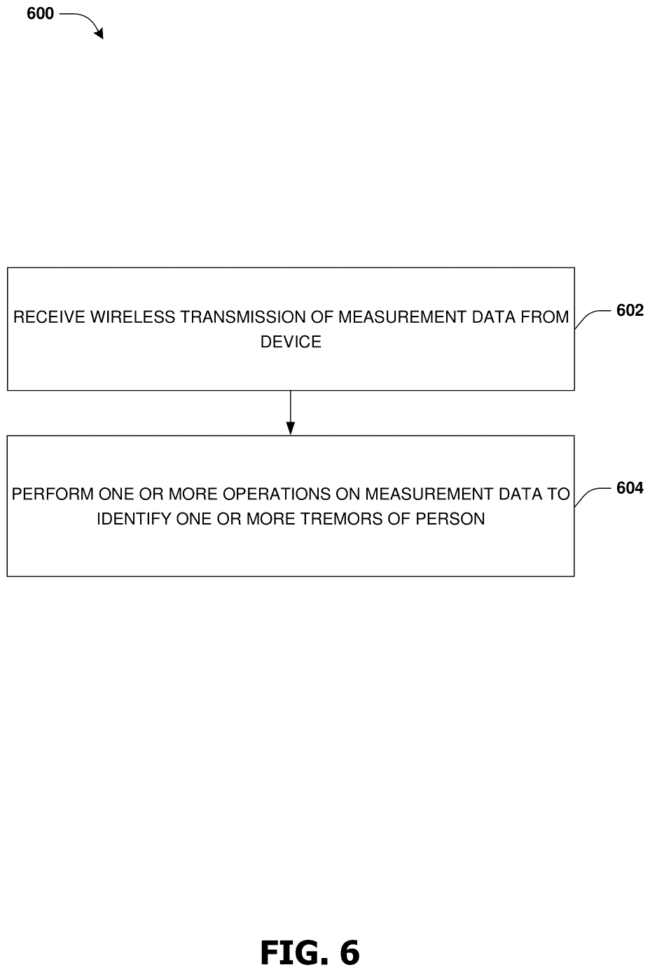

An embodiment of identifying one or more tremors of the person is illustrated by an example method 600 of FIG. 6. At 602, the first client device 450 may receive a wireless transmission of measurement data (e.g., the message 430) from the device 406. At 604, the first client device 450 may perform one or more operations on the measurement data to identify (and/or measure) one or more tremors of the person. In some examples, the first client device 450 may transmit content (e.g., the treatment response feedback 504) indicative of the one or more tremors to one or more computing devices (e.g., one or more computing devices of the treatment control system 506, such as at least one of a server of the treatment control system 506, an on-site client device, the remote client device, etc.).

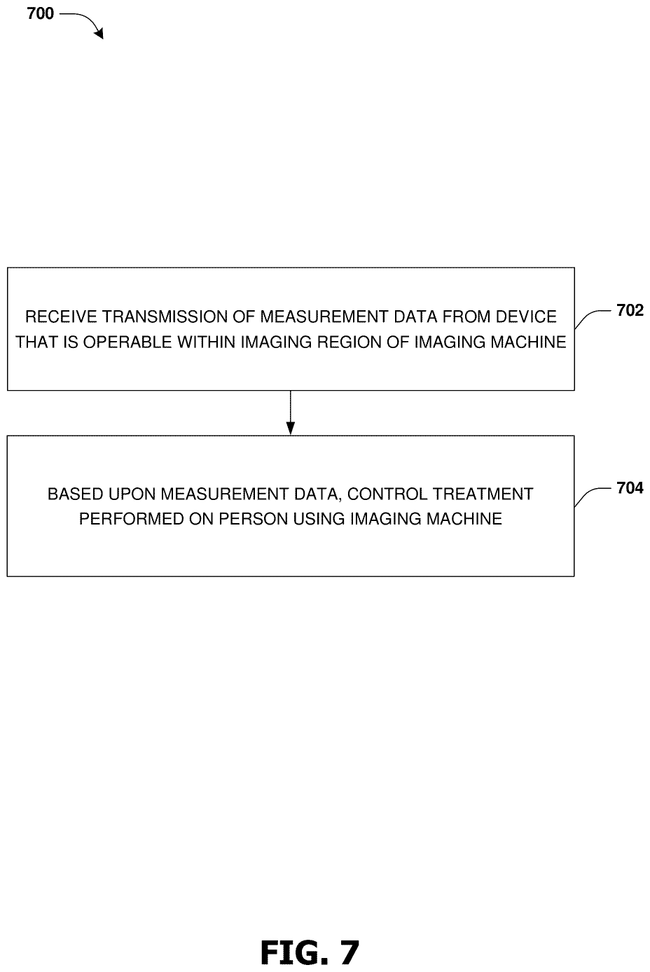

An embodiment of controlling the treatment of the person is illustrated by an example method 700 of FIG. 7. At 702, the first client device 450 may receive transmission of measurement data (e.g., the message 430) from a device (e.g., the device 406) that is operable within an imaging region of an imaging machine. At 704, the first client device 450 may control, based upon the measurement data, the treatment (e.g., the MRgFUS treatment) performed on the person using the imaging machine.

In some examples, the device 406 may be used in conjunction with one or more other tools (e.g., MR safe and/or MR conditional tools) to accurately capture a movement disorder of the person. In some examples, a keypad (e.g., a wireless keypad) that is operable within an imaging region of an imaging machine (e.g., at least one of MRI machine, CT scanning machine, ultrasound machine, etc.) used by a professional (e.g., a technician, a nurse, etc.) to control data acquisition of the device 406 and/or the first client device 450. For example, the professional may utilize the keypad to control the data acquisition while the professional and/or the keypad are within the MRI scanning room. For example, the keypad may be utilized (by the professional, for example) to submit timing markers about when accelerometer tests begin and/or finish. Alternatively and/or additionally, a display (e.g., a LCD, a touchscreen, a seven-segment display, a dot matrix display and/or other type of display) may be installed in the MRI scanning room and/or may display data (e.g., accelerometer results) determined using the device 406 so the professional (and/or other professionals) in the MRI scanning room may view (real-time) data produced using the device 406.

FIGS. 9A-9G illustrate a system 901. In some examples, the treatment system may comprise the system 901 and/or the system 501. In some examples, the system 901 may be used for facilitating a set of drafting tests (e.g., a set of one or more drafting tests) associated with the person. The one or more drafting tests may comprise (i) one or more first drafting tests performed prior to (and/or at a beginning of) the treatment, (ii) one or more second drafting tests performed during the treatment, and/or (iii) one or more third drafting tests (e.g., follow up drafting tests) performed after the treatment.

FIG. 9A illustrates a scenario in which a first drafting test associated with the person is performed. In some examples, a testing module of the system 901 may comprise a region of interest identification module 904 and/or a test result determination module 908. The testing module may identify an image 902 associated with the first drafting test. In some examples, the first drafting test may comprise one or more handwriting tests in which the person is tasked with handwriting at least one of text (e.g., a signature, a date, etc.), a symbol, etc. In an example shown in FIG. 9A, the first drafting test may comprise a first handwriting test in which the person is tasked with handwriting their signature. In some examples, the first drafting test may comprise one or more drawing tests in which the person is tasked with drawing at least one of a symbol, a picture, an object, etc. In an example shown in FIG. 9A, the first drafting test may comprise a first drawing test in which the person is tasked with drawing a spiral and/or a second drawing test in which the person is tasked with drawing a line. In an example, for the first drafting test, the person may make one or more markings on a sheet of paper (e.g., a drawing sheet with instructions and/or a template). The one or more markings may comprise (i) a signature draft 914 for the first handwriting test, (ii) a spiral draft 916 for the first drawing test, (iii) a line draft 918 for the second drawing test and/or (iv) one or more other markings. Embodiments are contemplated in which the one or more markings are made by the person using one or more tools other than a sheet of paper, such as using a touchpad, a touchscreen, etc. In some examples, the testing module may capture the image 902 using at least one of a camera, a scanner, etc. Alternatively and/or additionally, the testing module may receive the image 902 (from a device comprising at least one of a camera, a scanner, etc., for example).

In some examples, the testing module may analyze the image 902 to determine a first drafting test result 910 of the first drafting test. In an example, the region of interest identification module 904 may analyze the image 902 to identify (and/or extract) one or more regions of interest 906 corresponding to the one or more markings. For example, the one or more regions of interest 906 may comprise a first region of interest 920 corresponding to the signature draft 914, a second region of interest 922 corresponding to the spiral draft 916, and/or a third region of interest 924 corresponding to the line draft 918. In some examples, the region of interest identification module 904 may identify the one or more regions of interest 906 based upon a difference in at least one of color, thickness, etc. of the one or more markings in comparison with other features (e.g., test instructions printed on the sheet of paper). Alternatively and/or additionally, the one or more regions of interest 906 may be submitted to the test result determination module 908, which may determine the first drafting test result 910 based upon the one or more regions of interest 906.

In some examples, the first drafting test result 910 may comprise a first set of information associated with the first handwriting test. For example, the first set of information may comprise (i) a first total length of the signature draft 914 (e.g., a total length of ink, where increased wiggles of the signature draft 914 due to tremors may result in a greater value of the first total length of the signature draft 914), (ii) a first measure (e.g., quantity) of endpoints of the signature draft 914 (e.g., a measure of times a pen was lifted off the sheet of paper to produce the signature draft 914), (iii) a first measure (e.g., quantity) of crossing points of the signature draft 914 (e.g., a measure of loops and/or intersections of the signature draft 914), (iv) a first measure of curvature of the signature draft 914 (e.g., a mean radius of curvature along the signature draft 914, wherein a straight line may have an infinite radius of curvature and/or relatively fine wiggles may have a relatively smaller radius of curvature), which may be determined using a function for a parametrized line in two dimensions, and/or (v) a first handwriting score (e.g., a composite score of the first handwriting test), which may be determined based upon the first total length, the first measure of endpoints, the first measure of crossing points and/or the first measure of curvature indicated by the first set of information. In an example, the first handwriting score may correspond to log((totallength₁)×(endpoints₁−1)×(crossingpoints₁+1)×(curvature₁)), where totallength₁ may correspond to the first total length, endpoints₁ may correspond to the first measure of endpoints, crossingpoints₁ may correspond to the first measure of crossing points, and/or curvature₁ may correspond to the first measure of curvature. A higher value of the first handwriting score may represent increased tremor and/or a lower value of the first handwriting score may represent reduced tremor.

In some examples, the first drafting test result 910 may comprise a second set of information associated with the first drawing test. For example, the second set of information may comprise (i) a second total length of the spiral draft 916 (e.g., a total length of ink, where increased wiggles of the spiral draft 916 due to tremors may result in a greater value of the second total length of the spiral draft 916), (ii) a second measure (e.g., quantity) of endpoints of the spiral draft 916 (e.g., a measure of times a pen was lifted off the sheet of paper to produce the spiral draft 916), (iii) a second measure (e.g., quantity) of crossing points of the spiral draft 916 (e.g., a measure of loops and/or other intersections of the spiral draft 916), (iv) a second measure of curvature of the spiral draft 916 (e.g., a mean radius of curvature along the spiral draft 916), which may be determined using a function for a parametrized line in two dimensions, and/or (v) a first drawing score (e.g., a composite score of the first drawing test), which may be determined based upon the second total length, the second measure of endpoints, the second measure of crossing points and/or the second measure of curvature indicated by the second set of information. In an example, the first drawing score may correspond to log((totallength₂)×(endpoints₂−1)×(crossingpoints₂+1)×(curvature₂)), where totallength₂ may correspond to the second total length, endpoints₂ may correspond to the second measure of endpoints, crossingpoints₂ may correspond to the second measure of crossing points, and/or curvature₂ may correspond to the second measure of curvature. A higher value of the first drawing score may represent increased tremor and/or a lower value of the first drawing score may represent reduced tremor.

In some examples, the first drafting test result 910 may comprise a third set of information associated with the second drawing test. For example, the third set of information may comprise (i) a third total length of the line draft 918 (e.g., a total length of ink, where increased wiggles of the line draft 918 due to tremors may result in a greater value of the third total length of the line draft 918), (ii) a third measure (e.g., quantity) of endpoints of the line draft 918 (e.g., a measure of times a pen was lifted off the sheet of paper to produce the line draft 918), (iii) a third measure (e.g., quantity) of crossing points of the line draft 918 (e.g., a measure of loops and/or other intersections of the line draft 918), (iv) a third measure of curvature of the line draft 918 (e.g., a mean radius of curvature along the line draft 918), which may be determined using a function for a parametrized line in two dimensions, and/or (v) a second drawing score (e.g., a composite score of the second drawing test), which may be determined based upon the third total length, the third measure of endpoints, the third measure of crossing points and/or the third measure of curvature indicated by the third set of information. In some examples, a ratio may be determined by comparing the third total length with a length of a corresponding straight line. A logarithm scale of the (increased) ratio may be used as the second drawing score (to improve sensitivity to small changes, for example). For example, the second drawing score being 0 may indicate that the third total length was 1% larger than the length of the corresponding straight line. The second drawing score being 1 may indicate that the third total length was 10% larger than the length of the corresponding straight line. The second drawing score being 2 may indicate that the third total length was 100% larger than the length of the corresponding straight line. A higher value of the second drawing score may represent increased tremor and/or a lower value of the second drawing score may represent reduced tremor.

In some examples, the first drafting test result 910 may be displayed via an interface (e.g., at least one of the movement assessment interface, the treatment control interface 525, etc.) (for display to one or more healthcare professionals, such as physician, surgeon, nurse, etc., for example). Alternatively and/or additionally, the first drafting test result 910 may be used for controlling (e.g., advising, influencing, modifying, tailoring, aiding, facilitating, etc.) the treatment of the person (e.g., the treatment control system 506 may generate the treatment control signal 508 and/or the set of updated treatment parameters based upon the first drafting test result 910). For example, the treatment of the person may be controlled based upon the first drafting test result 910 using one or more of the techniques provided herein with respect to controlling the treatment based upon measurement data and/or movement features determined using the device 406. In some examples, at least some of the acts shown in and/or described with respect to FIG. 9A (e.g., at least one of capturing the image 902, identifying the one or more regions of interest 906, determining the first drafting test result 910, displaying the first drafting test result 910, etc.) may be performed using an application (e.g., a web application) installed on a client device (e.g., a phone, a tablet, a laptop, a computer, a wearable device, a smart device, a television, any other type of computing device, hardware and/or software), such as at least one of the first client device 450, a remote client device, an on-site client device, etc.

FIGS. 9B-9E illustrate data structures associated with drafting test results of drafting tests of the person. In some examples, one, some and/or all of the drafting test results and/or the drafting tests may be determined and/or performed using one, some and/or all of the techniques provided herein with respect to determining the first drafting test result 910 and/or performing the first drafting test. Each of the data structures shown in FIGS. 9B-9E has a horizontal axis corresponding to drafting tests of the person. For example, a horizontal axis value “pre” may correspond to a drafting test that is performed (i) prior to the treatment of the person and/or (ii) when the person is in an upright position. Horizontal axis values “1”, “2”, “3,” “4”, “5”, “6”, “7”, and/or “8” correspond to drafting tests performed (i) during the treatment (to guide the treatment, for example) and/or (ii) when the person is in a supine position. In an example in which the treatment comprises the MRgFUS treatment, the horizontal axis values “1”, “2”, “3,” “4”, “5”, “6”, “7”, and/or “8” correspond to drafting tests in association with (e.g., during and/or following) respective sonications of the treatment. For example, the horizontal axis values “1”, “2”, “3,” “4”, “5”, “6”, “7”, and/or “8” correspond to drafting tests in association with sonications “1”, “2”, “3,” “4”, “5”, “6”, “7”, and/or “8” of the treatment (e.g., horizontal axis value “1” corresponds to a drafting test performed in association with sonication “1” of the treatment, horizontal axis value “2” corresponds to a drafting test performed in association with sonication “2” of the treatment, etc.). In some examples, a sonication of the treatment may comprise emitting (using the ultrasound device 510, for example) one or more ultrasound energy beams to a target region of the person's body (e.g., a target region of the person's brain) (to perform an ablation on the target region, for example). Horizontal axis values “3 m”, “6 m”, and/or “12 m” correspond to follow up drafting tests performed after the treatment (to guide the treatment, for example), such as around 3 months after the treatment, around 6 months after the treatment and/or around 12 months after the treatment, respectively. For example, the person may perform the follow up drafting tests remotely, and/or may mail a physical copy of the follow up drafting tests to a healthcare provider and/or may capture and/or transmit images (e.g., the image 902) of the follow up tests to the healthcare provider (via at least one of email, an application, etc.).

FIG. 9B illustrates data structures associated with total lengths of drafting tests of the person. For example, the data structures include (i) a data structure 940 indicative of total lengths of signature drafts (which may include the first total length of the signature draft 914) derived from handwriting tests of the drafting tests, (ii) a data structure 942 indicative of total lengths of spiral drafts (which may include the second total length of the spiral draft 916) derived from spiral drawing tests of the drafting tests, and/or (iii) a data structure 944 indicative of total lengths of line drafts (which may include the third total length of the line draft 918) derived from line drawing tests of the drafting tests.

FIG. 9C illustrates data structures associated with measures of endpoints of drafting tests of the person. For example, the data structures include (i) a data structure 946 indicative of measures of endpoints of signature drafts (which may include the first measure of endpoints of the signature draft 914) derived from handwriting tests of the drafting tests, (ii) a data structure 948 indicative of measures of endpoints of spiral drafts (which may include the second measure of endpoints of the spiral draft 916) derived from spiral drawing tests of the drafting tests, and/or (iii) a data structure 950 indicative of measures of endpoints of line drafts (which may include the third measure of endpoints of the line draft 918) derived from line drawing tests of the drafting tests.

FIG. 9D illustrates data structures associated with measures of crossing points of drafting tests of the person. For example, the data structures include (i) a data structure 952 indicative of measures of crossing points of signature drafts (which may include the first measure of crossing points of the signature draft 914) derived from handwriting tests of the drafting tests, (ii) a data structure 954 indicative of measures of crossing points of spiral drafts (which may include the second measure of crossing points of the spiral draft 916) derived from spiral drawing tests of the drafting tests, and/or (iii) a data structure 956 indicative of measures of crossing points of line drafts (which may include the third measure of crossing points of the line draft 918) derived from line drawing tests of the drafting tests.

FIG. 9E illustrates data structures associated with measures of curvature of drafting tests of the person. For example, the data structures include (i) a data structure 958 indicative of measures of curvature of signature drafts (which may include the first measure of curvature of the signature draft 914) derived from handwriting tests of the drafting tests, (ii) a data structure 960 indicative of measures of curvature of spiral drafts (which may include the second measure of curvature of the spiral draft 916) derived from spiral drawing tests of the drafting tests, and/or (iii) a data structure 962 indicative of measures of curvature of line drafts (which may include the third measure of curvature of the line draft 918) derived from line drawing tests of the drafting tests.

In some examples, one or more representations of data comprising at least some data of one, some and/or all of the data structures shown in FIGS. 9B-9E (and/or other data such as handwriting scores, drawing scores, and/or other composite scores indicated by drafting test results) may be displayed (during the treatment, for example) via an interface (e.g., at least one of the movement assessment interface, the treatment control interface 525, etc.) for display to one or more healthcare professionals (e.g., physician, surgeon, nurse, etc.), which may (i) allow the one or more healthcare professionals to evaluate the degree of tremor symptoms of the person and/or determine whether the tremor symptoms are improving or worsening (in real time, for example), and/or (ii) provide guidance for treating the person. For example, after performing a sonication of the treatment (e.g., sonication 2) in which a third target region (of the person's brain, for example) is treated, a healthcare professional may view the one or more representations of data to determine whether tremor symptoms of the person improved or worsened compared with prior tremor symptoms of the person at a time prior to the sonication (e.g., the prior tremor symptoms may correspond to tremor symptoms of the person prior to the treatment and/or tremor symptoms of the person during and/or following sonication 1 of the treatment). The healthcare professional may decide to continue treating the third target region based upon a determination that the tremor symptoms of the person improved. The healthcare professional may decide to adjust the target region (e.g., switch from the third target region to a fourth target region) based upon a determination that the tremor symptoms of the person worsened (or did not improve).

FIG. 9F illustrates a tremor status module 964 determining a first tremor status 966 of the person based upon the first drafting test result 910 and/or accelerometer-derived data 963 determined using the device 406. For example, the accelerometer-derived data 963 may comprise at least some of the message 430 and/or one or more movement features (e.g., one or more tremor features) determined based upon the message 430. In some examples, the accelerometer-derived data 963 may be based upon data collected by the device 406 over a first period of time (when the person is in the testing position, for example), and the first drafting test may be performed over a second period of time. The first time period may at least partially overlap with the second period of time, or may be separate from the second period of time. In some examples, the first tremor status 966 may be indicative of a tremor severity level of the person. In some examples, an indication of the first tremor status 966 may be displayed via an interface (e.g., at least one of the movement assessment interface, the treatment control interface 525, etc.) (for display to one or more healthcare professionals, such as physician, surgeon, nurse, etc., for example). Alternatively and/or additionally, the first tremor status 966 may be used for controlling (e.g., advising, influencing, modifying, tailoring, aiding, facilitating, etc.) the treatment of the person (e.g., the treatment control system 506 may generate the treatment control signal 508 and/or the set of updated treatment parameters based upon the first tremor status 966). In some examples, determining the first tremor status 966 using the first drafting test result 910 and/or accelerometer-derived data 963 (in comparison with merely using a drafting test result or merely using accelerometer-derived data) may provide for (i) improved accuracy of the first tremor status 966 (which may provide a more accurate representation of the tremor severity level of the person), (ii) more accurate control of the treatment, and/or (iii) improved results of the treatment.

In some examples, tremor statuses of the person at various times may be determined. The tremor statuses may comprise (i) one or more first tremor statuses of the person prior to (and/or at a beginning of) the treatment, (ii) one or more second tremor statuses of the person during the treatment, and/or (iii) one or more third tremor statuses (e.g., follow up tremor statuses) of the person after the treatment.

FIG. 9G illustrate a data structure 970 representative of the tremor statuses of the person, which may include a tremor status 972a of the person prior to the treatment and/or tremor statuses 972b-972i of the person at one or more times during the treatment. In some examples, one, some and/or all of the tremor statuses may be determined using one, some and/or all of the techniques provided herein with respect to determining the first tremor status 966. In an example, the tremor status 972a may be determined based upon (i) a drafting test result of a drafting test performed prior to and/or at the beginning of the treatment and/or (ii) accelerometer-derived data based upon data collected by the device 406 prior to and/or at the beginning of the treatment (e.g., collected at a time when the person is determined to have the testing position). Alternatively and/or additionally, (A) the tremor status 972b may be determined based upon (i) a drafting test result of a drafting test performed during and/or following sonication 1 of the treatment and/or (ii) accelerometer-derived data based upon data collected by the device 406 during and/or following sonication 1 (e.g., collected at a time when the person is determined to have the testing position), (B) the tremor status 972c may be determined based upon (i) a drafting test result of a drafting test performed during and/or following sonication 2 of the treatment and/or (ii) accelerometer-derived data based upon data collected by the device 406 during and/or following sonication 2 (e.g., collected at a time when the person is determined to have the testing position), etc. In some examples, a higher value of a tremor status (which may range from 0 to 100, for example) may correspond to a greater tremor severity level of the person.

In some examples, a representation of at least some of the data structure 970 (and/or other data) may be displayed (during the treatment, for example) via an interface (e.g., at least one of the movement assessment interface, the treatment control interface 525, etc.) for display to one or more healthcare professionals (e.g., physician, surgeon, nurse, etc.), which may (i) allow the one or more healthcare professionals to evaluate the degree of tremor symptoms of the person and/or determine whether the tremor symptoms are improving or worsening (in real time, for example), and/or (ii) provide guidance for treating the person.

In some examples, one or more tremor statuses of the data structure 970 may be used (by the tremor status module 964 and/or the treatment control system 506, for example) to control the treatment. In some examples, in response to a determination that tremor symptoms of the person worsened from sonication 2 to sonication 3 (which may be derived from the tremor status 972d associated with sonication 3 being greater than the tremor status 972c associated with sonication 2), a fourth target region targeted by the ultrasound device 510 in sonication 3 may be switched (by the tremor status module 964 and/or the treatment control system 506, for example) to a fifth target region to be targeted via sonication 4. In some examples, in response to a determination that tremor symptoms of the person improved from sonication 3 to sonication 4 (which may be derived from the tremor status 972e associated with sonication 4 being less than the tremor status 972d associated with sonication 3), the fifth target region targeted by the ultrasound device 510 in sonication 4 may be targeted in sonication 5.

Thus, in accordance with some embodiments, the present disclosure provides techniques for objectively quantifying and/or measuring tremor symptoms of the person in multiple ways, which may be used to more accurately control the treatment of the person.

Although some examples provided herein relate to assessing movements and/or tremors of the person's arm, embodiments are contemplated in which the disclosed techniques are used for assessing movements and/or tremors of one or more other body parts (e.g., at least one of the person's chest, shoulder, head, neck, midsection, stomach, leg, foot, one or more joints, one or more regions of the person's body that are adjacent to and/or surround one or more joints, etc.) and/or one or more other types of tremors (in addition or as an alternative to movements and/or tremors of the person's arm), such as vocal tremors, tremors of other regions of the upper extremity of the person and/or tremors of one or more other regions of the person. Alternatively and/or additionally, the disclosed techniques may be used for re-animation of a paralytic arm associated with a brain injury. For example, the re-animation may be achieved via white matter tract and/or cortical stimulation using focused ultrasound (using the ultrasound device 510, for example). Alternatively and/or additionally, the disclosed techniques may be used for finding “live wires” and/or other regions that may be used to restore function after stroke and/or brain injury.

In some examples, at least a portion of the device 406 (e.g., a neck area of a turtleneck, a neck strap, etc.) may be proximal the neck and/or vocal cords of the person and/or may support an accelerometer in a position that enables the accelerometer to gather measurement data usable to identify one or more vocal tremors, and thus can be used for treating the person for vocal tremors (via MRgFUS, for example), tracking the person's vocal tremors over a period of time (e.g., one or more hours, one or more days, one or more months, etc.), etc. using the techniques provided herein.

In some examples, at least a portion of the device 406 (e.g., a torso area and/or upper body area of a shirt) may be proximal a chest, shoulder area and/or torso area of the person and/or may support an accelerometer in a position that enables the accelerometer to gather measurement data usable to identify one or more upper body tremors, and thus can be used for treating the person for upper body tremors (via MRgFUS, for example), tracking the person's upper body tremors over a period of time (e.g., one or more hours, one or more days, one or more months, etc.), etc. using the techniques provided herein.

Embodiments are contemplated in which one or more accelerometers of the set of accelerometers are supported in one or more respective positions along the person's body via an adhesive and/or other mechanism (in addition to and/or as an alternative to using the sleeve 404 and/or clothing to support an accelerometer in a desired position, for example). In some examples, a first accelerometer of the set of accelerometers may be positioned proximal a first portion of a body part of the person via a first adhesive (e.g., a sticking surface, glue, etc.) and/or a first band and/or a second accelerometer of the set of accelerometers may be positioned proximal a second portion of a body part of the person via a second adhesive (different than or the same as the first adhesive) and/or a second band (different than or the same as the first band).

It may be appreciated that one or more of the techniques provided herein may provide for increased healthcare coverage across more geographical areas, such as due, at least in part, to enabling movements and/or tremors of patients to be monitored and/or tracked remotely, and/or enabling the treatment (e.g., the MRgFUS treatment) to be performed remotely. For example, a patient (anywhere in the world, for example) might find an MRI equipped with a FUS unit but elect to have a remote clinic perform his/her treatment. As the volume of patients and/or data expand, the treatment system (and/or the machine learning model) may update and treatments may become faster, safer, and/or better in quality. In some examples, a healthcare provider may be unlocked from a particular time and/or a particular place/location, and/or the disclosed subject matter may provide for centralization of care with local personnel to include and/or need (only, for example) a nurse and/or a radiology technician (without a physician and/or other provider needing to be locally present at the geographical location of the patient, for example).

According to some embodiments, a device is provided. The device includes one or more accelerometers configured to generate measurement data based upon movement associated with a body part of a person; and a communication device configured to transmit a message comprising the measurement data to a computing device, wherein the device is operable within an imaging region of an imaging machine.

According to some embodiments, the one or more accelerometers comprise a first accelerometer positioned proximal a first portion of the body part via at least one of a first adhesive, a first band or a first sleeve; and/or a second accelerometer positioned proximal a second portion of the body part via at least one of a second adhesive, the first band or the first sleeve.

According to some embodiments, the communication device comprises a wireless communication device configured to transmit the message over a wireless connection or a wired communication device configured to transmit the message over a wired connection.

According to some embodiments, the imaging machine comprises at least one of a magnetic resonance imaging (MRI) machine, a computed tomography (CT) scanning machine, or an ultrasound machine.

According to some embodiments, the communication device is configured to transmit the measurement data during a treatment performed on the person using the imaging machine.

According to some embodiments, the computing device is configured to: identify one or more tremors based upon the measurement data; display a movement assessment interface comprising content indicative of the one or more tremors; display a treatment control interface for controlling one or more treatment parameters of the treatment; receive one or more updated treatment parameters via the treatment control interface; and control the treatment based upon the one or more updated treatment parameters.

According to some embodiments, the computing device is configured to: transmit content indicative of at least some of the measurement data to a remote network associated with a remote medical treatment site; receive, after transmitting the content, one or more updated treatment parameters from the remote network; and control the treatment based upon the one or more updated treatment parameters.

According to some embodiments, the computing device is configured to: update one or more treatment parameters based upon the measurement data to determine one or more updated treatment parameters; and control the treatment based upon the one or more updated treatment parameters.

According to some embodiments, the measurement data is indicative of: a displacement level of a tremor associated with the body part; an acceleration of the tremor; a frequency of the tremor; an angular velocity of the tremor; an angle of the tremor; and/or a direction of motion associated with the tremor.

According to some embodiments, the body part corresponds to an arm of the person. The one or more accelerometers comprise: a first accelerometer proximal a wrist of the arm; a second accelerometer proximal a finger of the arm; a third accelerometer proximal a forearm of the arm; and/or a fourth accelerometer proximal a region, of the arm, between a shoulder of the person and an elbow of the arm.

According to some embodiments, the message is usable to control one or more machines performing an imaging-based treatment on the person.

According to some embodiments, the measurement data is indicative of: a displacement level of the movement; an acceleration of the movement; a frequency of the movement; and/or a direction associated with the movement.

According to some embodiments, a system is provided. The system comprises the device and a testing module configured to: at least one of capture or receive an image associated with a first drafting test associated with the person, wherein the first drafting test comprises at least one of a drawing test or a handwriting test; and analyze the image to determine a first drafting test result comprising at least one of a measure of line length of the first drafting test, a measure of endpoints of the first drafting test, a measure of crossing points of the first drafting test, a measure of curvature of the first drafting test, or a drafting score of the first drafting test. The system comprises a tremor status module configured to determine a tremor status of the person based upon at least one of the first drafting test result or the measurement data; and at least one of: display an indication of the tremor status; or control, based upon the tremor status, a treatment performed on the person using the imaging machine.

According to some embodiments, a computer-implemented method is provided. The computer-implemented method comprises receiving a wireless transmission of measurement data from a wearable device; and performing one or more operations on the measurement data to identify one or more tremors of a person.

According to some embodiments, the computer-implemented method comprises transmitting content indicative of the one or more tremors to one or more computing devices.

According to some embodiments, the computer-implemented method comprises displaying, via a movement assessment interface, content indicative of the one or more tremors; displaying a treatment control interface for controlling one or more treatment parameters; receiving one or more updated treatment parameters via the treatment control interface; and controlling a treatment of the person based upon the one or more updated treatment parameters.

According to some embodiments, the computer-implemented method comprises transmitting content indicative of the one or more tremors to a remote network associated with a remote medical treatment site; receiving, after transmitting the content, one or more updated treatment parameters from the remote network; and controlling a treatment of the person based upon the one or more updated treatment parameters.

According to some embodiments, the computer-implemented method comprises updating one or more treatment parameters based upon the measurement data to determine one or more updated treatment parameters; and controlling a treatment of the person based upon the one or more updated treatment parameters.

According to some embodiments, the computer-implemented method comprises: using a machine learning model trained using historical treatment data to at least one of (i) update an ongoing treatment plan based upon the measurement data to determine an updated version of the ongoing treatment plan, or (ii) generate a recommended treatment plan based upon the measurement data; and at least one of: controlling a treatment of the person based upon at least one of the ongoing treatment plan or the recommended treatment plan; or displaying a representation of at least one of the updated version of the ongoing treatment plan or the recommended treatment plan.

According to some embodiments, the wearable device is operable within an imaging region of a magnetic resonance imaging (MRI) machine; receiving the wireless transmission is performed during an MRI-based treatment performed on the person using the MRI machine; and the computer-implemented method comprises determining one or more updated treatment parameters based upon the one or more tremors; and controlling the MRI-based treatment based upon the one or more updated treatment parameters.

According to some embodiments, a computing device is provided. The computing device comprises a processor; and memory comprising processor-executable instructions that when executed by the processor cause performance of operations. The operations comprise receiving a wireless transmission of measurement data from a device; and performing one or more operations on the measurement data to identify one or more tremors of a person.

According to some embodiments, the device is operable within an imaging region of a magnetic resonance imaging (MRI) machine; receiving the wireless transmission is performed during an MRI-based treatment performed on the person using the MRI machine; and the operations comprise determining one or more updated treatment parameters based upon the one or more tremors; and controlling the MRI-based treatment based upon the one or more updated treatment parameters.

According to some embodiments, a method is provided. The method comprises receiving a transmission of measurement data from a device that is operable within an imaging region of an imaging machine; and based upon the measurement data, controlling a treatment performed on a person using the imaging machine.

According to some embodiments, controlling the treatment comprises: selecting a target region based upon the measurement data; and emitting, using an ultrasound device, one or more ultrasound energy beams to the target region.

According to some embodiments, controlling the treatment comprises: selecting an energy level based upon the measurement data; and emitting, using an ultrasound device, one or more ultrasound energy beams having the energy level to a region.

According to some embodiments, controlling the treatment comprises: selecting a target region and/or an energy level based upon the measurement data; and emitting, using an ultrasound device and based upon the target region and/or the energy level, one or more ultrasound energy beams, wherein the one or more ultrasound energy beams are targeted towards the target region and/or the one or more have the energy level.

According to some embodiments, the imaging machine comprises at least one of a magnetic resonance imaging (MRI) machine, a computed tomography (CT) scanning machine, or an ultrasound machine.

According to some embodiments, a device is provided. The device comprises one or more accelerometers configured to generate measurement data based upon movement associated with a body part of a person; and an indicator device configured to output a treatment indication based upon the measurement data, wherein the treatment indication is usable to control a treatment of the person.

According to some embodiments, the device is operable within an imaging region of at least one of a magnetic resonance imaging (MRI) machine, a computed tomography (CT) scanning machine, or an ultrasound machine.

According to some embodiments, the indicator device comprises a display and/or a set of light sources; and the treatment indication comprises a visual indication produced using at least one of the display or the set of light sources.

According to some embodiments, the indicator device comprises a speaker; and the treatment indication comprises audio produced using the speaker.

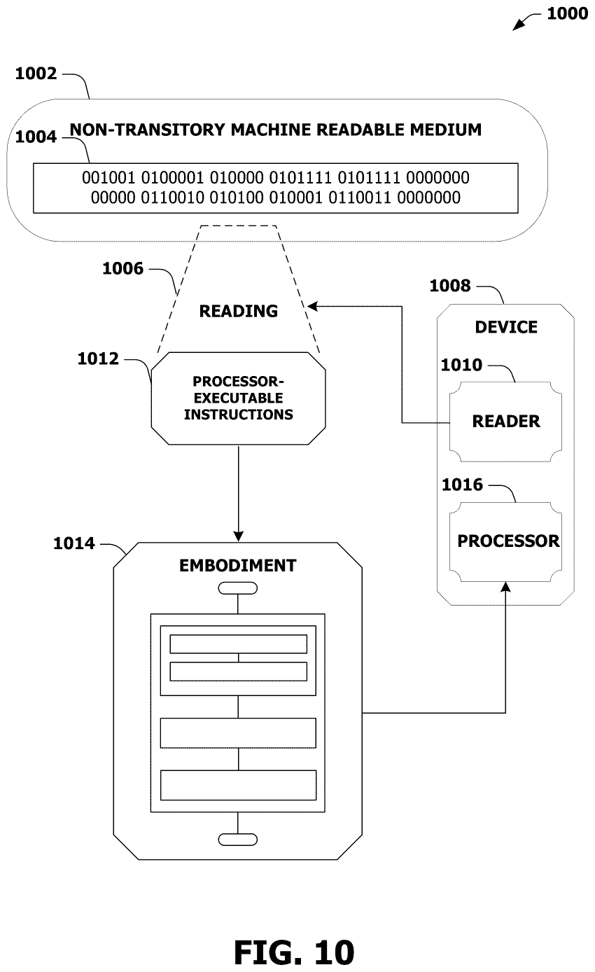

FIG. 10 is an illustration of a scenario 1000 involving an example non-transitory machine readable medium 1002. The non-transitory machine readable medium 1002 may comprise processor-executable instructions 1012 that when executed by a processor 1016 cause performance (e.g., by the processor 1016) of at least some of the provisions herein (e.g., embodiment 1014).

The non-transitory machine readable medium 1002 may comprise a memory semiconductor (e.g., a semiconductor utilizing static random access memory (SRAM), dynamic random access memory (DRAM), and/or synchronous dynamic random access memory (SDRAM) technologies), a platter of a hard disk drive, a flash memory device, or a magnetic or optical disc (such as a compact disc (CD), digital versatile disc (DVD), or floppy disk).

The example non-transitory machine readable medium 1002 stores computer-readable data 1004 that, when subjected to reading 1006 by a reader 1010 of a device 1008 (e.g., a read head of a hard disk drive, or a read operation invoked on a solid-state storage device), express the processor-executable instructions 1012.

In some embodiments, the processor-executable instructions 1012, when executed, cause performance of operations, such as at least some of the example method 600 of FIG. 6 and/or the example method 700 of FIG. 7, for example. In some embodiments, the processor-executable instructions 1012 are configured to cause implementation of a system, such as at least some of the example system 501 of FIGS. 5A-5N, the treatment system, the treatment control system 506 and/or the example system 901 of FIGS. 9A-9G, for example.

As used in this application, “component,” “module,” “system”, “interface”, and/or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Various operations of embodiments are provided herein. In an embodiment, one or more of the operations described may constitute computer readable instructions stored on one or more computer and/or machine readable media, which if executed will cause the operations to be performed. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein. Also, it will be understood that not all operations are necessary in some embodiments.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A device, comprising:
one or more accelerometers configured to generate measurement data based upon movement associated with a body part of a person; and
a communication device configured to transmit a message comprising the measurement data to a computing device, wherein the device is operable within an imaging region of an imaging machine.

2. The device of claim 1, wherein the one or more accelerometers comprise at least one of:
a first accelerometer positioned proximal to a first portion of the body part via at least one of a first adhesive, a first band or a first sleeve; or
a second accelerometer positioned proximal to a second portion of the body part via at least one of a second adhesive, the first band or the first sleeve.

3. The device of claim 1, wherein the communication device comprises:
a wireless communication device configured to transmit the message over a wireless connection; or
a wired communication device configured to transmit the message over a wired connection.

4. The device of claim 1, wherein the imaging machine comprises at least one of a magnetic resonance imaging (MRI) machine, a computed tomography (CT) scanning machine, or an ultrasound machine.

5. The device of claim 1, wherein:
the communication device is configured to transmit the measurement data during a treatment performed on the person using the imaging machine.

6. The device of claim 5, wherein:
the computing device is configured to:
identify one or more tremors based upon the measurement data;
display a movement assessment interface comprising content indicative of the one or more tremors;
display a treatment control interface for controlling one or more treatment parameters of the treatment;
receive one or more updated treatment parameters via the treatment control interface; and
control the treatment based upon the one or more updated treatment parameters.

7. The device of claim 5, wherein:
the computing device is configured to:
transmit content indicative of at least some of the measurement data to a remote network associated with a remote medical treatment site;
receive, after transmitting the content, one or more updated treatment parameters from the remote network; and
control the treatment based upon the one or more updated treatment parameters.

8. The device of claim 5, wherein:
the computing device is configured to:
update one or more treatment parameters based upon the measurement data to determine one or more updated treatment parameters; and
control the treatment based upon the one or more updated treatment parameters.

9. The device of claim 1, wherein the measurement data is indicative of at least two of:
a displacement level of a tremor associated with the body part;
an acceleration of the tremor;
a frequency of the tremor;
an angular velocity of the tremor;
an angle of the tremor; or
a direction of motion associated with the tremor.

10. The device of claim 1, wherein:
the body part corresponds to an arm of the person; and
the one or more accelerometers comprise at least one of:
a first accelerometer proximal a wrist of the arm;
a second accelerometer proximal a finger of the arm;
a third accelerometer proximal a forearm of the arm; or
a fourth accelerometer proximal a region, of the arm, between a shoulder of the person and an elbow of the arm.

11. The device of claim 1, wherein the message is usable to control one or more machines performing an imaging-based treatment on the person.

12. A system comprising:
the device of claim 1; and
a testing module configured to:
at least one of capture or receive an image associated with a first drafting test associated with the person, wherein the first drafting test comprises at least one of a drawing test or a handwriting test; and
analyze the image to determine a first drafting test result comprising at least one of:
a measure of line length of the first drafting test;
a measure of endpoints of the first drafting test;
a measure of crossing points of the first drafting test;
a measure of curvature of the first drafting test; or
a drafting score of the first drafting test; and
a tremor status module configured to:
determine a tremor status of the person based upon at least one of the first drafting test result or the measurement data; and
at least one of:
display an indication of the tremor status; or
control, based upon the tremor status, a treatment performed on the person using the imaging machine.

13. A computer-implemented method, comprising:
receiving a wireless transmission of measurement data from a wearable device; and
performing one or more operations on the measurement data to identify one or more tremors of a person.

14. The computer-implemented method of claim 13, comprising:
transmitting content indicative of the one or more tremors to one or more computing devices.

15. The computer-implemented method of claim 13, comprising:
displaying, via a movement assessment interface, content indicative of the one or more tremors;
displaying a treatment control interface for controlling one or more treatment parameters;
receiving one or more updated treatment parameters via the treatment control interface; and
controlling a treatment of the person based upon the one or more updated treatment parameters.

16. The computer-implemented method of claim 13, comprising:
transmitting content indicative of the one or more tremors to a remote network associated with a remote medical treatment site;
receiving, after transmitting the content, one or more updated treatment parameters from the remote network; and
controlling a treatment of the person based upon the one or more updated treatment parameters.

17. The computer-implemented method of claim 13, comprising:
using a machine learning model trained using historical treatment data to at least one of:
update an ongoing treatment plan based upon the measurement data to determine an updated version of the ongoing treatment plan; or
generate a recommended treatment plan based upon the measurement data; and
at least one of:
controlling a treatment of the person based upon at least one of the ongoing treatment plan or the recommended treatment plan; or
displaying a representation of at least one of the updated version of the ongoing treatment plan or the recommended treatment plan.

18. The computer-implemented method of claim 13, wherein:
the wearable device is operable within an imaging region of a magnetic resonance imaging (MRI) machine;
receiving the wireless transmission is performed during an MRI-based treatment performed on the person using the MRI machine; and
the computer-implemented method comprises:
determining one or more updated treatment parameters based upon the one or more tremors; and
controlling the MRI-based treatment based upon the one or more updated treatment parameters.

19. A method comprising:
receiving a transmission of measurement data from a device that is operable within an imaging region of an imaging machine; and
based upon the measurement data, controlling a treatment performed on a person using the imaging machine.

20. The method of claim 19, wherein controlling the treatment comprises:
selecting at least one of a target region or an energy level based upon the measurement data; and
emitting, using an ultrasound device and based upon at least one of the target region or the energy level, one or more ultrasound energy beams, wherein at least one of:
the one or more ultrasound energy beams are targeted towards the target region; or
the one or more ultrasound energy beams have the energy level.
